(19) United States
(12) Patent Application Publication
(10) Pub. No.: US 2019/0269718 A1
(43) Pub. Date: Sep. 5, 2019

(54) METHODS FOR ADVERSELY AFFECTING BIOFILMS

(71) Applicants: Commonwealth Scientific and Industrial Research Organisation, Campbell (AU); Monash University, Clayton (AU)

(72) Inventors: Katherine Elizabeth Sarah Locock, Malvern East (AU); Laurence Meagher, Brunswick (AU); Yue Qu, Bundoora (AU); Ana Traven, Alphington (AU)

(73) Assignees: Commonwealth Scientific and Industrial Research Organisation, Campbell (AU); Monash University, Clayton (AU)

(21) Appl. No.: 16/410,665

(22) Filed: May 13, 2019

Related U.S. Application Data
(62) Division of application No. 15/555,247, filed on Sep. 1, 2017.

(30) Foreign Application Priority Data
Mar. 2, 2015 (AU) 2015900721

(51) Int. Cl. A61K 31/785; A01N 47/44; A61L 15/24; A61L 15/44; A61L 27/34; A61L 29/16; A61L 29/08; A61L 27/54

(52) U.S. Cl. CPC A61K 31/785; A01N 47/44; A61L 15/24; A61L 15/44; A61L 2300/404; A61L 29/16; A61L 29/085; A61L 27/54; A61L 27/34

(57) ABSTRACT

The present invention relates to a method of adversely affecting a biofilm, the method comprising exposing the biofilm to a composition comprising an effective amount of polymer that incorporates within its backbone structure a first polymerised residue of ethylenically unsaturated monomer, said first polymerised monomer residue comprising a covalently bound moiety that (i) presents pendant from the backbone structure, and (ii) comprises a cationic functional group or precursor functional group thereof.

### FIELD OF THE INVENTION

The present invention relates in general to biofilms. In particular, the invention relates to a method of adversely affecting a biofilm.

### BACKGROUND OF THE INVENTION

A biofilm is an established aggregation or community of microorganisms that adhere to each other on a biotic or abiotic surface or at an interface and, together with a matrix of extracellular polymeric substance (EPS) secreted by the microorganisms (sometimes referred to in the art as “slime”), form a film structure.

Human infections associated with biofilms are highly correlated to serious life-threatening disease. This is due to the recalcitrance of biofilms to conventional antimicrobial agents, and also lower efficiency of the immune system in clearing these infections.

Common microorganisms implicated in biofilm formation include Gram-positive bacteria such as Staphylococcus aureus, Coagulase-negative Staphylococcus, and Streptococcus sp., Gram-negative bacteria such as Klebsiella pneumoniae and Pseudomonas aeruginosa, and a fungal pathogen such as Candida sp.

Biofilm related infections outnumber those caused by planktonic microorganisms. The fungus Candida albicans; and the bacteria Staphylococcus aureus and Staphylococcus epidermidis, are among the most common causes of medical device-associated infections.

According to the Center for Disease Control and Prevention (CDC)/The National Healthcare Safety Network (NHSN) Reports, Staphylococcus aureus and Candida albicans are the most prevalent bacterial and fungal pathogens causing Healthcare Acquired Infections (HAIs) such as catheter-related bloodstream infections.

Bloodstream infections caused by Candida species and Staphylococcus aureus are often associated with high overall mortality. The presence of a biofilm formed from these types of microorganisms on the surface of a medical device that is provided to an immuno-compromised patient can pose a high risk factor for mortality of that patient.

In a clinical situation, biofilm related infections can be mono- or polymicrobial, and can occur at multiple sites of a subject. Approximately 20%-40% of the cases of candidaemia were reportedly accompanied by bacteremia, with Staphylococcus species being the predominant accompanying pathogens, and the trend is rising.

In polymicrobial biofilms, the microbial species can benefit each other. Beneficial interactions have been observed between S. aureus and C. albicans, such as higher microorganism load and increased antimicrobial resistance of the polymicrobial growth mode relative to single species biofilms. For example, when growing in polymicrobial biofilms with C. albicans, S. aureus becomes more resistant to vancomycin and daptomycin than as a monoculture.

Another bacterial species, Staphylococcus epidermidis, has been shown to protect C. albicans from the action of the antifungal drugs fluconazole and amphotericin B when growing together in polymicrobial biofilms. The enhanced drug resistance in polymicrobial biofilms has been attributed to components of the EPS secreted by fungi and bacteria. It is thought that the major culprit in biofilm drug resistance are the carbohydrates in the extracellular matrix, for example 1,3 β-glucan, through sequestration of the antimicrobial compounds.

Other extracellular components have also been implicated in antimicrobial resistance. A fragment of the cell surface mucin Msb2 that is secreted by C. albicans contributes to daptomycin resistance of S. aureus in polymicrobial biofilms through direct binding. Mechanisms of inter-species cross-talk could also contribute to increased resistance to therapy, such as quorum sensing pathways, and bacterial, cell wall components affecting C. albicans morphogenesis and biofilm formation.

Growth as a C. albicans-S. aureus polymicrobial biofilm has been found to result in an altered innate immune response, with elevated proinflammatory cytokines and influx of neutrophils that were not able to control the infection, compared with the monomicrobial infection.

With significant negative human health and related cost implications associated with biofilms, there is an urgent socioeconomic need for developing anti-microbial agents that can effectively and efficiently treat biofilms.

### SUMMARY OR THE INVENTION

The present invention therefore provides a method of adversely affecting a biofilm, the method comprising exposing the biofilm to a composition comprising an effective amount of polymer that incorporates within its backbone structure a first polymerised residue of ethylenically unsaturated monomer, said first polymerised monomer residue comprising a covalently bound moiety that (i) presents pendant from the backbone structure, and comprises a cationic functional group or precursor functional group thereof.

Surprisingly, polymer used in accordance with the invention has advantageously been found to function as a potent anti-microbial agent and exert an adverse affect on biofilms. For example, exposing a biofilm to the polymer has been found to kill microorganisms that are embedded in and/or form part of the biofilm. Polymers used in accordance with the invention have been found to outperform the efficacy of conventional antifungal and antibacterial drugs for treating biofilms, particularly in an in vitro or in vivo setting. Furthermore, use of polymer in accordance with the invention advantageously has been found to be highly effective at killing microorganisms that are embedded in and/or form pan of polymicrobial biofilms, for example polymicrobial biofilms comprising Candida albicans and Staphylococcus aureas.

For convenience, the polymer used in accordance with the invention may be referred to herein as an anti-microbial or anti-biofilm agent.

In one embodiment, polymer used in accordance with the invention further incorporates within its backbone structure a second polymerised residue of ethylenically unsaturated monomer, said second polymerised monomer residue comprising a covalently bound hydrophobic moiety that presents pendant from the backbone structure.

Where the polymer used in accordance with the invention comprises both the first and second polymerised residues, the invention may therefore be described in terms of a method of adversely affecting an established biofilm, the method comprising contacting the biofilm with a composition comprising an effective amount of polymer that incorporates within its backbone structure first and second polymerised residues of ethylenically unsaturated monomer, said first polymerised monomer residue comprising a covalently bound moiety that (i) presents pendant from the backbone structure, and (ii) comprises a cationic functional group or a precursor functional group thereof, said second polymerised monomer residue comprising a covalently bound hydrophobic moiety that presents pendant from the backbone structure.

In one embodiment, polymer used in accordance with the invention is a copolymer. The copolymer may be a random, alternating, block or statistical copolymer.

The biofilm which is adversely affected according to the present invention may be a monomicrobial or polymicrobial biofilm. The method according to the invention has surprisingly been found to be effective at adversely affecting polymicrobial biofilms.

In one embodiment, the biofilm is a polymicrobial biofilm.

In another embodiment, the biofilm is a polymicrobial biofilm comprising Gram-positive bacterium and a fungal pathogen.

In a further embodiment, the polymer (used in accordance with the invention) is the only anti-microbial agent to which the biofilm is exposed. In other words, in one embodiment the biofilm is exposed to no other antimicrobial agent other than the polymer (used in accordance with the invention).

Accordingly, in one embodiment the composition comprises no other antimicrobial agent other than the polymer (used in accordance with the invention).

A biofilm adversely affected according to the present invention may comprise, or consist essentially of, one or more microorganisms selected from Gram-positive bacteria such as Staphylococcus aureus, Staphylococcus epidermidis, Coagulase-negative Staphylococcus, Streptococcus sp., and mycobacterium tuberculosis. Gram-negative bacteria such as Klebsiella pneumoniae and Pseudomonas aeruginosa, and fungal pathogens such as Candida sp., and Candida albicans.

In one embodiment, the biofilm adversely affected according to the present invention comprises, or consists essentially of, one or both microorganisms selected from Candida albicans and Staphylococcus aureus.

The method according to the invention may be particularly effective for adversely affecting a biofilm located on or in a subject. In that case, the present invention further provides a method of adversely affecting a biofilm located on or in a subject, the method comprising exposing the biofilm to a composition comprising an effective amount of polymer by administering the composition to the subject, wherein the polymer incorporates within its backbone structure a first polymerised residue of ethylenically unsaturated monomer, said first polymerised monomer residue comprising a covalently bound moiety that (i) presents pendant from the backbone structure, and (ii) comprises a cationic functional group or a precursor group thereof.

The present invention also provides a composition suitable for administration to a subject on or in which is located a biofilm, the composition comprising a pharmacologically acceptable carrier and an effective amount of polymer that incorporates within its backbone structure a first polymerised residue of ethylenically unsaturated monomer, said first polymerised monomer residue comprising a covalently bound moiety that (i) presents pendant from the backbone structure, and (ii) comprises a cationic functional group or a precursor functional group thereof.

The composition according to the invention will typically be administered to a subject having an infectious disease, condition or disorder associated with, characterised by, or caused by the presence of a biofilm in or on the subject.

Accordingly, the present invention also provides a composition suitable for administration to a subject when used in the treatment of an infectious disease, condition or disorder associated with, characterised by, or caused by the presence of a biofilm in or on the subject, the composition comprising a pharmacologically acceptable carrier and polymer that incorporates within its backbone structure a first polymerised residue of ethylenically unsaturated monomer, said first polymerised monomer residue comprising a covalently bound moiety that (i) presents pendant from the backbone structure, and (ii) comprises a cationic functional group or precursor functional group thereof.

The present invention further provides use of polymer in the manufacture of a medicament for adversely affecting a biofilm, the polymer incorporating within its backbone structure a first polymerised residue of ethylenically unsaturated monomer, said first polymerised monomer residue comprising a covalently bound moiety that (i) presents pendant from the backbone structure, and (ii) comprises a cationic functional group or a precursor functional group thereof.

The present invention also provides use of polymer in the manufacture of a medicament for the treatment of an infectious disease, condition or disorder associated with, characterised by or caused by the presence of a biofilm in or on a subject, the polymer incorporating within its backbone structure a first polymerised residue of ethylenically unsaturated monomer, said first polymerised monomer residue comprising a covalently bound moiety that (i) presents pendant from the backbone structure, and (ii) comprises a cationic functional group or a precursor functional group thereof.

In one embodiment, the manufactured medicament comprises the polymer and a pharmacologically acceptable carrier.

In another embodiment, the polymer is used in the manufacture of a medicament for killing microorganisms that form part of a biofilm located on or in a subject.

Compositions comprising polymer according to the invention have advantageously been found to function as an effective and efficient lock solution.

Accordingly, the present invention further provides a method of performing antimicrobial lock therapy on a medical device having a biofilm adhered thereto, the method comprising exposing the biofilm to a composition comprising an effective amount of polymer that incorporates within its backbone structure a first polymerised residue of ethylenically unsaturated monomer, said first polymerised monomer residue comprising: a covalently bound moiety that (i) presents pendant from the backbone structure, and (ii) comprises a cationic functional group or precursor functional group thereof.

The present invention further provides an antimicrobial lock solution for use in antimicrobial lock therapy, the composition comprising a pharmacologically acceptable carrier and a polymer that incorporates within its backbone structure a first polymerised residue of ethylenically unsaturated monomer, said first polymerised monomer residue comprising a covalently hound moiety that (i) presents pendant from the backbone structure, and (ii) comprises a cationic functional group or precursor functional group thereof.

Further aspects and embodiments of the invention described in more detail below.

### BRIEF DESCRIPTION OF THE DRAWINGS

Embodiments of the invention are described herein with reference to the following non-limiting drawings in which:

FIG. 1 illustrates the structure of a polymer suitable for use according to the invention in the form of guanylated polymethacrylates PG3 and PG4. The polymers were synthesised from the monomers 2-guanidinoethyl methacrylate (2-GEMA) and methyl methacrylate (MMA);

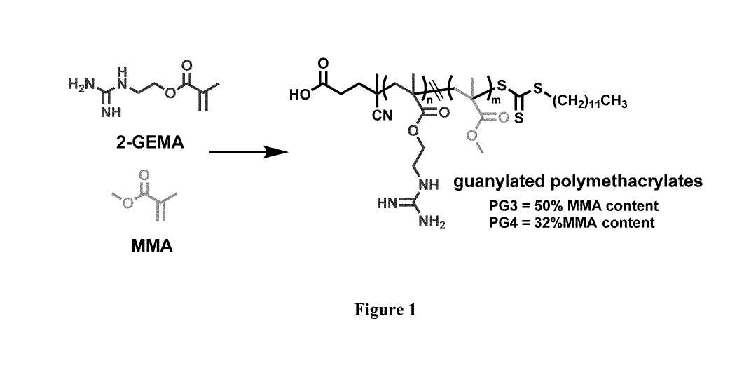

FIG. 2 illustrates that fungus-derived extracellular matrix assists S. aureus association into polymicrobial biofilms. (A) Scanning electron microscopy (SEM) of mixed S. aureus-C. albicans biofilms. Scale bar=5 μm. For SEM of mono-culture biofilms see FIG. 6A. (B) SEM of S. aureus mono-culture biofilms grown in the presence or absence of extracellular matrix derived from C. albicans biofilms. (C) S. aureus CFUs in polymicrobial biofilm formed by C. albicans strain DAY 185 wild type or bgl2ΔΔ mutant strains were determined using viable count quantification, as described in the Examples section. The number of bacterial cells was expressed relative to the number of C. albicans cells in the biofilm. Error bars represent standard deviation from three independent biological repeats performed in triplicate:

FIG. 3 illustrates that polymer according to the invention is efficient at killing polymicrobial C. albicans-S. aureus biofilm. CLSM reconstructions of S. aureus and C. albicans single and polymicrobial biofilm treated with different antimicrobial agents. CLSM reconstructions show the 3D staining pattern for live cells (SYTO-9, green) and dead cells (PI, red). Biofilms formed by S. aureus and/or C. albicans established over a twenty-four hour period were exposed to antimicrobial agents prepared in RPMI-1640 for 18 hours and then stained with BacLight Live/Dead Viability kit (3.35 uM SYTO-9 and 20 uM propidium iodide). The experiments were performed twice and representative images are shown:

FIG. 4 illustrates polymer according to the invention outperforms current antimicrobial-drug combinations in eradicating mixed fungal -bacterial biofilms. Survival of cells in S. aureus-C. albicans polymicrobial biofilms upon antimicrobial exposure. Log % survival is a logarithm format of the percentage of surviving cells for statistical analysis. For example, log % survival of −2 stands for approximately 1 % (10⁻²) of the original population persisting following antimicrobial treatment. The lower the log % survival is, the belter established biofilm-killing efficacy of the antimicrobial agents. The experiment was done with 3 biological repeats in duplicate, shown are averages and the standard deviation;

FIG. 5 illustrates the role for extracellular matrix in the susceptibility of C. albicans biofilms to polymer according to the invention. Efficacy of guanylated polymethacrylates against monomicrobial biofilms formed by C. albicans DAY 185 wild type and bgl2ΔΔ mutant was tested by treatment of the established biofilms overnight in the presence or absence of PG3. PG4 or fluconazole as control. The percentage of remaining established biofilm was calculated by dividing the CPUs of established biofilms exposed to drugs with the CPUs of biofilms exposed to drug-free growth medium. The experiment was done with 3 biological repeats in triplicate, shown are averages and the standard deviation;

FIG. 6 illustrates an assessment of the polymicrobial biofilm formation (A) Scanning electron microscopy of single biofilms of either C. albicans and S. aureus. Scale bar=5 82 m. (B) Semi-quantitative CV assay shows S. aureus and C. albicans synergistically forms single and polymicrobial biofilms in RPMI-1640. Error bars represent standard deviation; and

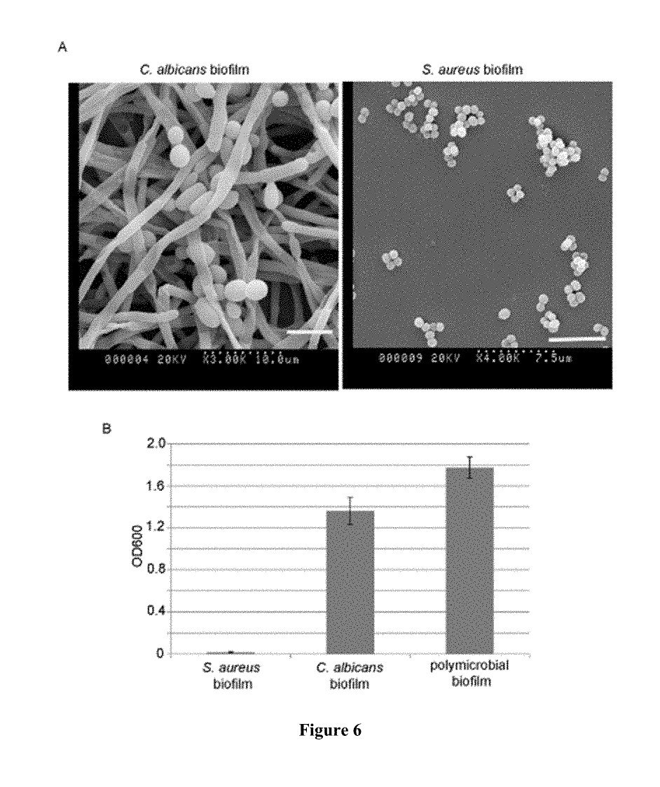

FIG. 7 illustrates the gene expression in wild type and bgl2 mutant established biofilms. RT-PCR revealed no difference between the WT and bgl2 mutant biofilms in the expression level of ALS3, ALS1, HWP1, EAP1 and BCR1, encoding C. albicans surface-associated adhesins and a transcriptional regulator of biofilm formation.

### DEFINITIONS

To facilitate an understanding of the present invention, a number of terms and expressions are defined below. Other terms and expressions are also defined later in the document.

The articles “a” and “an” are used herein to refer to one or to more than one (i.e. to at least one) of the grammatical object of that article For example, reference to “a microorganism” is intended to mean one microorganism or more than one microorganism.

The term “about” used herein is understood to be a reference to a range of numbers that a person skilled in the art would consider equivalent to the recited value in the context of achieving the same functional result.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “biofilm” refers to an established aggregation or community of microorganisms that adhere to each other on a biotic or abiotic surface or at an interface and, together with a matrix of extracellular polymeric substance (EPS) secreted by the microorganisms (sometimes referred to in the art as “slime”), form a film-like structure. For avoidance of any doubt, the term “biofilm” is not intended to be reference to a mere microorganism cluster or microorganisms in a planktonic slate. Those skilled in the art can readily detect the presence of an established biofilm using known techniques.

Reference herein to a biofilm “containing” a particular microorganism is intended to mean that the biofilm may comprise that microorganism(s), consists essentially of that microorganism(s) (i.e. the microorganism(s) in question is the predominant species or type of microorganism(s) of the biofilm), or consist of that microorganism(s) (i.e. the microorganism(s) in question is the only species or type of microorganism(s) of the biofilm).

Reference herein to the term “microorganism”, or associated terms such as “microbial” and “microbial organism”, is intended to mean any organism that exists as a microscopic cell that is included within the domains of archaea bacteria or eukarya. Accordingly, the tern) is intended to encompass prokaryotic or eukaryotic cells or organisms having a microscopic size and includes bacteria, archaea and eubacteria of all species as well as eukaryotic microorganism such as yeast and fungi.

The expression “biofilm forming microorganism” therefore refers to any microorganism that is capable of forming a biofilm, including monomicrobial and polymicrobial biofilms.

By “adversely affecting” a biofilm, or a biofilm being “adversely affected” is intended to mean that the viability of the biofilm is compromised in some way. For example, a biofilm will be adversely affected if the number of live microorganisms that form part of the biofilm is reduced. A biofilm may also be adversely affected if its growth is inhibited, supressed, retarded or prevented.

According to one embodiment of the invention, the expression “adversely affecting” a biofilm is intended to mean killing microorganisms that form part of the biofilm.

Microorganisms that “form part of” a biofilm may be located at a surface of, or embedded within, the biofilm.

As used herein the expression “anti-microbial agent” is intended to mean any agent that alone, or in combination with another agent, is capable of killing or inhibiting the growth of one or more species of microorganisms.

Reference herein to “exposing” the biofilm to the composition of polymer used in accordance with the invention is intended to mean bringing the biofilm into contact with the composition or polymer. Exposing the biofilm to the composition or polymer may be achieved by any suitable means and includes application of the composition or polymer to the biofilm and administration of the composition or polymer to a subject comprising the biofilm. In this context, the terms “exposing”, “administering” and “contact” and grammatical variations thereof may be used to interchangeably throughout the specification.

As used herein the expression “effective amount” refers to the amount of a substance (e.g. a composition comprising polymer used in accordance with the invention) sufficient to achieve a desired result, which in the case of the present invention is to adversely affect a biofilm. The exact amount required to achieve the desired result will vary depending on various factors such as a subject or a situation under consideration, the composition of the biofilm, the volume or size of the biofilm to be exposed to the composition, the environment in which the biofilm is located and the means by which exposing the biofilm to the composition is conducted. An effective amount can be provided for in one or more applications, administrations or dosages and is not intended to be limited to a particular formulation, administration route or application method. Accordingly, it is not practical to specify an exact “effective amount”. Taking into account the particular circumstances, a person skilled in the art could readily determine the “effective amount” through routine experimentation.

Reference herein to a “subject” should be understood to encompass humans, primates (e.g. monkeys), livestock animals (e.g. sheep, pigs, cattle, horses, donkeys and goats), laboratory test animals (e.g. mice, rabbits, rats and guinea pigs), companion animals (e.g. dogs and cats) and captive wild animals (e.g. lions, tigers, zebra, kangaroos and deer). In the context of the invention, the term “subject” will generally refer to a human or non-human animal who will receive, or who has received, treatment for a condition caused by a biofilm.

It should be understood that the terms “treatment”, “treat” and “treating” do not necessarily imply that a subject is treated until total recovery from a particular condition. Accordingly, treatment includes reducing the severity of an existing condition, amelioration of the symptoms of a particular condition or preventing or otherwise reducing the risk of developing a particular condition.

As used herein the term “administration” refers to the act of giving a drug, prodrug, or other agent, or therapeutic treatment (e.g. a composition or polymer used in accordance with the present invention) to a physiological system (e.g. a subject or invivo, invitro, exvivo cells, tissue and organs). Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. The composition or polymer used in accordance with the invention may also be administered or applied directly to the biofilm.

The expressions “pharmaceutically acceptable” or “pharmacologically acceptable”, as used herein, refer to compositions that do not substantially produce adverse reactions, for example toxic, allergic or analogical reactions, when administered to a subject.

The expression “pharmaceutically acceptable” or “pharmacologically acceptable” carrier or composition is intended to be a substance suitable for administration to a subject in its own right. In other words, administration of the carrier per se, the polymer used in accordance with the invention, the composition according to the invention comprising a carrier and any other constituent component of the composition, to a subject will not result in unacceptable toxicity, including allergenic responses and disease states. The carrier may be a liquid, gel or solid substrate.

As a guide only, a person skilled in the art may consider “pharmaceutically acceptable” or “pharmacologically acceptable” as an entity approved by a regulatory agency of a Federal or State government or listed in the US Pharmacopeia or other generally recognised pharmacopeia for use in animals, and more particularly humans. Having said this, those skilled in the art will appreciate that the suitability of a composition for administration to a subject and whether or not a given substance would be considered pharmacologically acceptable will to some extend depend upon the mode of administration selected. Thus, the mode of administration may need to be considered when evaluating whether a given composition is suitable for administration to a subject or pharmacologically acceptable.

Examples of pharmacologically acceptable liquid carriers include, but are not limited to, phosphate buffered saline solution, water, emulsions (for example water in oil or oil in water emulsions), ethanol, polyol (for example glycerol, propylene glycol and liquid polyethylene glycol, and the like), vegetable oils, and combinations thereof.

Examples of pharmacologically acceptable solid carriers include, but are not limited to, metals, ceramics, plastics, textiles and combinations thereof.

Other suitable pharmacologically acceptable substances that may be used include stabilisers and adjuvants.

As used herein, the term “medical device” includes any material or device that is used on, in, or through a subjects' body. Such use may, for example, be in the course of medical treatment of the subject for a particular disease, condition or injury. Medical devices include, but are not limited to items such as implants, wound care devices and drug delivery devices. Examples of medical implants include urinary catheters, intravascular catheters, dialysis shunts, wound drain tubes, skin sutures, vascular grafts, implantable measures, intraocular devices, heart valves and the like. Examples of wound care devices include, but are not limited to, wound dressings, biological graft materials, tape closures and dressings, and surgical incise drapes. Examples of drug delivery devices include, but are not limited to, needles, drug delivery skin patches, drug delivers mucosal patches and medical sponges.

A composition according to the invention may be or form part of a medical device.

### DETAILED DESCRIPTION OF THE INVENTION

The method according to the invention comprises exposing a biofilm to the composition described herein. The biofilm is adversely affected upon being exposed to the composition. The biofilm may be adversely affected by its growth being prevented or inhibited, or by the number of viable microorganisms that form part of the biofilm being reduced. In other words, the composition used in accordance with the invention is cytostatic or cytotoxic to the biofilm.

By the composition being cytostatic to the biofilm is meant that microorganisms that form the biofilm are prevented or inhibited from growth or replication.

By the composition being cytotoxic to the biofilm is meant the number of viable microorganisms that form part of the biofilm is reduced (e.g. where microorganisms that form part of the biofilm are killed).

In one embodiment, the invention therefore provides a method of killing microorganisms that form pan of a biofilm, the method comprising features as outlined herein.

The composition used in accordance with the invention may present as the polymer per se or the polymer may be provided in combination with a suitable a carrier. The carrier may be a pharmacologically acceptable carrier. The carrier may be a liquid, gel or solid substrate. When the carrier is in the form of a liquid or gel, the polymer will typically be dissolved or dispersed in the liquid or gel. When the carrier is in the form of a solid substrate, the polymer may be absorbed within the substrate and/or form a coating on the substrate. The solid substrate may be porous (i.e. contain voids or holes).

The composition used in accordance the invention comprises an effective amount of polymer that incorporates within its backbone structure a first polymerised residue of ethylenically unsaturated monomer.

The expression “polymerised residue of ethylenically unsaturated monomer” is intended to mean the reaction residue formed as a result of free radical polymerisation of ethylenically unsaturated monomer which provides for the polymer chain. It is the ethylenically unsaturated group of the monomer that actively participates in the free radical polymerisation reaction. Those skilled in the art will appreciate that such a monomer reaction will provide for a carbon-carbon segment or residue (—C—C—) within the backbone structure of the so formed polymer chain. Polymer used in accordance with the invention may therefore be described as an addition polymer (i.e. not a condensation polymer).

Polymer used in accordance with the invention incorporates a first polymerised monomer residue comprising a covalently bound moiety that (i) presents pendant from the backbone. structure, and (ii) comprises a cationic functional group or a precursor functional group dimwit. The moiety will therefore be covalently bound and in pendant formation to the —C—C— segment. The cationic functional group or a precursor functional group thereof will typically be at least one atom removed from the backbone structure (i.e. there will be at least one atom between the carbon based backbone structure of the polymer chain and the pendant cationic functional group or a precursor functional group thereof). Accordingly, the covalently bound moiety may (i) present pendant from the backbone structure, and (ii) comprise a cationic functional group or a precursor functional group thereof that is at least one atom (or at least two or three atoms) removed from the backbone structure.

Polymer used in accordance with the invention may be simplistically illustrated as comprising structure (A), where [symbol] represents the remainder of the polymer backbone, C—C represents the carbon-carbon segment derived from the first polymerised residue of ethylenically unsaturated monomer, and “------” represents the covalent attachment of atoms to the carbon-carbon (C—C) segment of the moiety (CAT) in pendant formation. The moiety (CAT) will typically be at least one atom removed from the backbone structure. The polymer will generally incorporate with its backbone structure a plurality of first polymerised monomer residues.

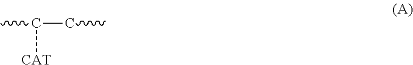

In addition to the first polymerised residue of ethylenically unsaturated monomer, polymer in accordance with the invention may comprise a second polymerised residue of ethylenically unsaturated monomer. The second polymerised monomer residue comprises a covalently bound hydrophobic moiety that presents pendant from, the polymer backbone structure. The hydrophobic moiety will typically also be at least one atom removed from the backbone structure.

Where polymer used in accordance with the invention comprises both first and second polymerised residues of ethylenically unsaturated monomer, the polymer may be simplistically illustrated by structure (B), where in addition to features of structure (A) outlined above structure (B) further comprises a carbon-carbon segment (C—C) derived from the second polymerised residue of ethylenically unsaturated monomer to which is covalently attached the hydrophobic moiety (BIC) in pendant formation. The polymer (B) will generally incorporate within its backbone structure a plurality of both first and second polymerised monomer residues and as such it will be a copolymer, for example a random copolymer.

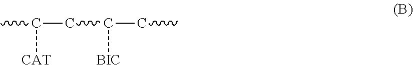

Polymer used in accordance with the invention therefore presents pendant from its backbone structure (i) a covalently bound moiety which comprises a cationic functional group or a precursor functional group thereof (CAT), and optionally (ii) a covalently bound hydrophobic moiety (BIC).

Those skilled in the art will appreciate that a cationic functional group hares a positive charge. A skilled person will also appreciate the range of functional groups that can present as a cation. For example, cationic functional groups will generally comprise a nitrogen and/or phosphorous atom.

When in the form of a cationic functional group, that fun functional group bares the positive charge. A “precursor functional group thereof” is therefore a functional group that typically presents in a neutral state and can be converted into a cation, for example through addition or removal of an electrophile. A “precursor functional group” is therefore typically neutral but chargeable to form a cation through, for example, pH dependant promotion (for example at physicological pH), or quaternisation, to afford the corresponding cationic functional group.

In one embodiment, the cationic functional group or precursor functional group thereof is selected from amine, phosphine and onium (e.g. ammonium and phosphonium) functional groups.

In a further embodiment the cationic functional group or precursor functional group thereof is selected from primary amine, secondary amine, tertiary amine, ammonium (i.e. quaternary amine) and phosphonium (i.e. quaternary phosphine) functional groups.

In another embodiment, the cationic functional group or precursor functional group thereof is. selected from guanidine and amidino.

The pendant and covalently bound hydrophobic moiety may be provided by suitable hydrophobic moieties known to those skilled in the art. For example, the hydrophobic moiety may be selected from alkyl, alkenyl, alkynyl, aryl, carbocyclyl, heterocyclyl, heteroaryl, alkylalkenyl, alkylalkynyl, alkylaryl, alkylcarbocyclyl, alkylheterocyclyl, alkylheteroaryl, alkylalkenylalkyl, alkylalkynylalkyl, alkylarylalkyl, alkylacylalkyl, arylakylaryl, arylalkenylaryl, arylalkynylaryl, arylcarbocyclyl, arylhetcrocyclyl and arylheteroaryl.

In a further embodiment, the covalently bound hydrophobic moiety is selected from C₁₋₁₈ alkyl and C₆₋₁₈ aryl.

Polymer use in accordance with the invention may be described such dust the first polymerised residue of ethylenically unsaturated monomer has a structure represented in general formula (I):

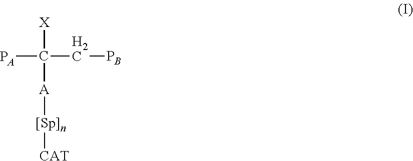

where
P_{A} and P_{B}, which are the same or different, represent the remainder of the polymer backbone structure;
X is selected from H and optionally substituted C₁-C₆ alkyl;
A is a moiety capable of activating an ethylenically unsaturated double bond such that it will undergo free radical polymerisation;
Sp is a spacer moiety;
n is 0 or 1; and
CAT is the moiety comprising a cationic functional group or precursor functional group thereof.

Similarly the polymer used in accordance with the invention may be described such that the second polymerised residue of ethylenically unsaturated monomer has a structure represented in general formula (II):

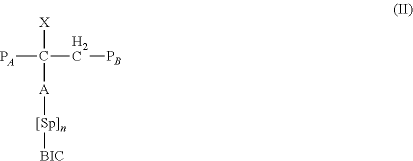

where:
P_{A} and P_{B}, which are the same or different, represent the remainder of the polymer backbone structure;
X is selected from H and optionally substituted C₁-C₆ alkyl;
A is a moiety capable of activating an ethylenically unsaturated double bond such that it will undergo free radical polymerisation;
Sp is a spacer moiety;
n is 0 or 1; and
BIC is the hydrophobic moiety.

Those skilled in the art will appreciate that reference to the first polymerised residue of ethylenically unsaturated moiety having a structure as “presented in general formula (I)” is intended to highlight the specific residue of general formula (I*):

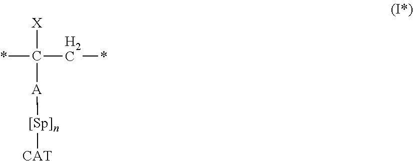

where X, A, Sp, CAT and a are as herein defined, and -* represents a covalent bond that will be coupled to an atom of the remainder of the polymer backbone structure or chain at *.

P_{A} and P_{B} are presented in general formula (1) to: (a) more clearly depict the polymer backbone structure, and (h) help illustrate the pendant formation of the covalently bound (CAT).

The first polymerised residue of ethylenically unsaturated monomer represented by general formula (I) illustrates how the moiety (CAT) is at least one atom removed from the polymer backbone structure (i.e. CAT is separated from the backbone structure by at least A).

Generally, polymer used in accordance with the invention will comprise multiple of such polymerised residues. In that case, each polymerised residue may be the same or different.

A similar rationale applies to the second polymerised residue of ethylenically unsaturated monomer having a structure as “presented in general formula (II)”.

The expression “incorporates within its backbone structure” in the context of polymer used in accordance with the invention is therefore intended to mean that the polymerised residue of ethylenically unsaturated monomer (e.g. that provides the —C—C— component of general formula (I)) forms together with the remainder of the polymer chain (e.g. P_{A} and P_{B} in general formula (I)) a string of atoms that are each connected so as to form the polymer chain which may be linear or branched. In other words, the polymerised residue of ethylenically unsaturated monomer per se is not pendant from the polymer backbone structure. However, it will be appreciated that the moiety comprising the cationic functional group or precursor functional group thereof will be pendant from the polymer backbone (as will be the hydrophobic moiety). For example, in the context of general formula (I), the first polymerised residue of ethylenically unsaturated monomer (—C—C—) in formula (I) is not pendant from the polymer backbone. However, the —A—[Sp]ₙ—CAT component of formula (I) is pendant from the polymer backbone structure. The moiety (CAT) is also at least one atom removed from the backbone structure.

In the general formulae described herein X is selected from H and optionally substituted C₁-C₆ alkyl. In one embodiment, X is selected from H and CH₃.

The divalent group “—A—” in the general formulae described herein is a moiety capable of activating an ethylenically unsaturated double bond such that it will undergo free radical polymerisation. Those skilled in the art will appreciate that in the context of general formulae (I) and (II) the polymerised residue of the ethylenically unsaturated moiety is the polymerised residue of a monomer of general formula (III), and as such there no longer remains in formulae (I) and (II) the ethylenically unsaturated double bond that requires activation to undergo polymerisation. Accordingly, the word “capable” is used in the context of formulae (I)-(III) to indicate a particular property of the A group and not a requirement that the ethylenically unsaturated double bond must be present per se (although it is of course present in formula (II)).

Those skilled in the an will appreciate ethylenically unsaturated double bonds typically require activation so that they are sufficiently reactive to take part in free radical polymerisation. Such activation is generally achieved by covalently coupling an activating group to the double bond within suitable proximity to promote sufficient activation.

Those skilled in the art will further appreciate the range of moieties A that are capable of activating an ethylenically unsaturated double bond such that it will undergo free radical polymerisation. Generally, A will be selected from an aromatic- or a heteroatom containing-moiety. For example, A may be selected from a carbonyl or carbonyl containing functional group such as an ester, an anhydride, an amide or an imide, an ether functional group, or an aromatic functional group such as a phonylene group.

In one embodiment, A is a divalent moiety selected from carbonyl, ether, ester, amide, anhydride, imide and optionally substituted arylene.

In a further embodiment, A is a divalent moiety selected from carbonyl (*—C(O)—), ether (*—O—) ester (*—O—C(O)—), amide (*—(R^{z})N—C(O)—), anhydride (*—C(O)—O—C(O)—), imide (*—C(O)—(R^{z})N—C(O)—) and optionally substituted arylene (*—Ar—), where R^{z} is or C₁-C₆ Ar is arylene, and * notates the point of coupling to Sp or CAT, BIC and M when n=0.

When n=0, A may form together with CAT a moiety capable of activating an ethylenically unsaturated double bond such that it will undergo free radical polymerisation. For example, when n=0, A may be a carbonyl moiety and CAT may comprise a —(R^{z})N— group (where R^{z} is H or C₁ C₆ alkyl) such that -A-CAT form together an amide functional group.

Accordingly, A may be defined as a moiety which, alone or in combination with CAT, BIC or M when n=0, is capable of activating an ethylenically unsaturated double bond such that it will undergo free radical polymerisation,

In one embodiment, when n=1, A may be a divalent moiety selected from carbonyl, ether, ester, amide, anhydride, imide and optionally substituted arylene.

in a further embodiment, when A may be a divalent moiety selected from carbonyl (*—C(O)—) ether (*—O—), ester (*—O—C(O)—), amide (*—(R^{z})N—C(O)—), anhydride (*—C(O)—O—C(O)—), imide (*—C(O)—(R^{z})N—C(O)—) and optionally substituted arylene (*—Ar—), where R^{z} is H or C₁-C₆ alkyl, Ar is arylene, and * notates the point of coupling to Sp.

The divalent group “—[Sp]ₙ—” in the general formulae defined herein is a spacer moiety. For avoidance of any doubt, when “n” is 0 it is intended that the spacer moiety Sp is absent from the formulae, and as such the moiety A is directly coupled to moiety CAT, BIC or M. Furthermore, when “n” is 1 it is intended that the spacer moiety is present in the general formulae, and as such the spacer moiety bridges moieties A and CAT, BIC or M. When present, the spacer moiety Sp may function to increase the distance of CAT, BIC or M from the backbone polymer structure.

There is no particular limitation regarding the nature of the spacer moiety Sp. Examples of suitable spacer moieties Sp include an optionally substituted divalent form of a group selected from alkyl, alkenyl, alkynyl, aryl, acyl, carbocyclyl, heterocyclyl, heteroaryl, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkyloxyacylalkyl, alkylthioalkyl, alkenythioalkyl, alkynylthioalkyl, arylthioalkyl, alkylalkenylalkyl, alkylalkynylalkyl, alkylarylalkyl, alkylacylalkyl, arylalkylaryl, arylalkenylaryl, arylalkynyaryl, arylacylaryl, arylatcyl, arylcabocyclyl, arylheterocyclyl, arylheteroaryl, alkenyloxyaryl, alkynloxyaryl, aryloxyaryl, alkyltbioaryl, alkenylthioaryl, alkynylthioaryl, arylthioaryl and polyoxyalkylene.

In lists herein defining groups from which a moiety or functional group may be selected, each alkyl, alkenyl, alkynyl, aryl, carbocycyl, heteroaryl, and heterocyclyl moiety may be optionally substituted. For avoidance of any doubt, where a given group contains two or more of such moieties (e.g. alkylaryl), each of such moieties may be optionally substituted with one, two, three Of more optional substituents as herein defined.

The term “polyoxyalkylene” used herein is intended to mean an oligomer or polymer built from oxyalkylene units. The polyoxyalkylene may be branched or linear.

When characterising as polyoxyalkylene, it can sometimes be convenient to refer to the number of oxyalkylene units that make up the polyoxyalkylene. A polyoxyalkylene used in accordance with the invention will generally comprise 2 to about 50, or from 2 to about 25 oxyalkylene units, or from 2 to about 15 oxyalkylene units.

In the context of a polyoxyalkylene, the term “oxyalkylene” used herein is intended to mean a divalent —O(CR^{X}R^{Y})ᵢ— group, where R^{X} and R^{Y} are each independently selected from hydrogen and optionally substituted alkyl, and i is an integer ranging from 1 to 10. Generally, R^{X} and R^{Y} are each independently selected from hydrogen and optionally substituted C₁₋₆ alkyl, and i is an integer selected from 2, 3, and 4. When i>1, each (CR^{X}R^{Y}) may be the same or different. For example, when the oxyalkylene unit is an oxyethylene unit, R^{X} and R^{Y} are both hydrogen and (i=2 (i.e. —O(CH₂)₂—), and where the oxyalkylene unit is an oxypropylene unit, i=2 and R^{X} and R^{Y} of the first “i” are both hydrogen and R^{X} and R^{Y} of the second “i” can respectively be hydrogen and methyl (i.e. —OCH₂CH(CH₃)—).

Each oxyalkylene group or unit within the polyoxyalkylene may be the same or different. In other words, the polyoxyalkylene may be a homopolymer or a copolymer (including a random or block copolymer). The oxyalkylene units may be derived from an alkylene oxide such as ethylene oxide, propylene oxide, or butyl one oxide.

Further examples of a suitable spacer moiety Sp include optionally substituted: C₁₋₁₈ alkyl, C₆₋₁₈ aryl, C₁₋₁₈ alkyl —C(O)—, C₆₋₁₈ aryl —C(O)—, and —(—O(CR^{X}R^{Y})ᵢ—)ᵢ—, where R^{X}, R^{Y}, i are as herein defined and r is an integer ranging from 2-15.

When n=1, Sp may form together with BIC a hydrophobic moiety. For example, when n=1, Sp and BIC may be alkyl such that -Sp-BIC form together the hydrophobic moiety.

As herein defined, -CAT comprises a cationic functional group of precursor functional group thereof.

In one embodiment, -CAT comprises one oi more atoms selected from nitrogen, phosphorous and combination thereof.

In a further embodiment, -CAT comprises a functional group selected from amine, phosphine, onium and combination thereof.

In another embodiment, -CAT comprises a functional group selected from primary amine, secondary amine, tertiary amine, ammonium, phosphonium and combinations thereof.

In yet a further embodiment, -CAT is selected from guanidine and amidino.

The pendant -BIC group may be provided by any suitable hydrophobic moiety known to those skilled in the an. For example, -BIC may be selected from alkyl, alkenyl, alkynyl, aryl, carbocyclyl, heterocyclyl, heteroaryl, alkylalkenyl, alkylalkynyl, alkylaryl, alkylcarbocyclyl, alkylheterocyclyl, alkyheteroaryl, alkylalkenylalkyl, alkylakynylalkyl, alkylarylalkyl, alkylacylalkyl, arylalkylaryl, arylalkenylaryl, arylalkynylaryl, arylcarbocyclyl, arylheterocyclyl and arylheteroaryl.

In one embodiment, -BIC is selected from C₁₋₁₈ alkyl and C₆₋₁₈ aryl.]

Polymer used in accordance with the invention may be a homopolymer or a copolymer. In the case of a homopolymer, those skilled in the art will appreciate that only one type of ethylenically unsaturated monomer will be used to prepare the polymer.

In the case of a copolymer, those skilled in the art will appreciate that two or more different ethylenically unsaturated monomers will be used to prepare the polymer.

In the case of a homopolymer, it will therefore be appreciated that P_{A} and P_{B} in general formulae (I) and (II) will be the same.

Ethylenically unsaturated monomer used to prepare the polymer used in accordance with the invention includes and may be represented by general formula (III):

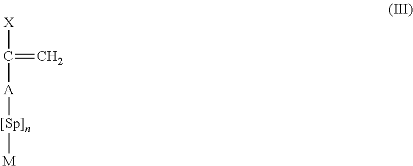

where:
X is selected from H and optionally substituted C₁-C₆ alkyl;
A is a moiety capable of activating an ethylenically unsaturated double bond such that it will undergo free radical polymerisation;
Sp is a spacer moiety;
n is 0 or 1; and
M is CAT or BIC as herein defined.

In one embodiment, M in formula (III) is CAT.

In another embodiment, M in formula (III) is BIC.

When M in formula (III) is CAT, the ethylenically unsaturated monomer will provide for the first polymerised residue represented in formula (I). Similarly, when M in formula (III) is BIC the ethylenically unsaturated monomer will provide for the second polymerised residue represented in general formula (II).

Polymer used in accordance with the invention will generally comprise multiple first polymerised residues represented in general formula (I). When present, the polymer will also generally comprise multiple second polymerised residues represented in general formula (II). In the context of general formulae (I) and (II), such multiple polymerised residues of the first and second ethylenically unsaturated monomers will typically form part or all of the polymer backbone structure represented by P_{A} and P_{B}.

Polymerised residues of ethylenically unsaturated monomer that form part of the polymer backbone structure of polymer used in accordance with the invention an typically funned through a free radical polymerisation process. Factors that determine the free radical (co)polymerisability of ethylenically unsaturated monomers are well documented in the art. For example, see: Greenlee, R. Z, in Polymer Handbook 3^{rd} edition (Brandup, J, and Immergut. E. H. Eds) Wiley: New York, 1989, p II/53.

As will be described in more detail below, polymer used in accordance with the invention that is in the form of a homopolymer may be prepared by polymerising monomer of the same. structure from formula (III) where M is CAT. Polymer used in accordance with the invention in the form of a copolymer may be prepared by polymerising monomers of different structure from formula when M is CAT, or by polymerising monomer of formula (III) where M is CAT with one or more other suitable copolymerisable ethylenically unsaturated monomers. Examples of suitable copolymerisable ethylenically unsaturated monomers will include those of general formula (III) when M is BIC and also other ethylenically unsaturated monomers of general formula (IV):

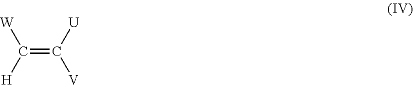

where U and W are independently selected from —CO₂H, —CO₂R¹, —COR¹, —CSR¹, —CSOR¹, —COSR¹, —CONH₂, —CONHR¹, —CONR¹₂, hydrogen, halogen and optionally substituted C₁-C₄ alkyl or U and W form together a lactone, anhydride or imide ring that may itself be optionally substituted, where the optional substituents are independently selected from hydroxy, —CO₂H, —CO₂R¹, —COR¹, —CSR¹, —CSOR¹, —COSR¹, —CN, —CONH₂, —CONHR¹, —CONR¹₂, —OR¹, —SR¹, —O₂CR¹, —SCOR¹, and —OCSR¹;
V is selected from hydrogen. R¹, —CO₂H, —CO₂R¹, —COR¹, —CSR¹, —COSR¹, —COSR¹, —CONR₂, —CONHR¹, —CONR¹₂, —OR¹, —SR¹, —O₂CR¹, —SCOR¹, and —OCSR¹;
where the or each R¹ is independently selected from optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted arylalkyl, optionally substituted heteroarylalkyl, optionally substituted alkylaryl, optionally substituted alkylheteroaryl, and an optionally substituted polymer chain.

The or each R¹ in formula (IV) may also be independently selected from optionally substituted C₁-C₂₂ alkyl, optionally substituted C₂-C₂₂ alkenyl, optionally substituted C₂-C₂₂ alkynyl, optionally substituted C₆-C₁₈ aryl, optionally substituted C₃-C₁₈ heteroaryl, optionally substituted C₃-C₁₈ carbocyclyl, optionally substituted C₂-C₁₈ heterocyclyl, optionally substituted C₇-C₂₄ arylalkyl, optionally substituted C₄-C₁₈ heteroarylalkyl, optionally substituted C₇ alkylaryl, optionally substituted C₄-C₁₈ alkylheteroaryl, and an optionally substituted polymer chain.

In one embodiment, R¹ (in formula (IV) may be independently selected from optionally substituted C₁-C₆ alkyl.

Examples of optional substituents for in formula (IV) include those selected from alkyleneoxidyl (epoxy), hydroxy, alkoxy, acyl, acyloxy, formyl, alkylcarbonyl, carboxy, sulfonic acid, alkoxy- or aryloxy-carbonyl, isocyanato, cyano, silyl, halo, amino, including salts and derivatives thereof. Examples polymer chains include those selected from polyalkyene oxide, polyaxylene ether and polyalkylene ether.

Examples of monomers of formula (IV) include maleic anhydride, N-alkylmaleimide, N-arylmaleimide, dialkyl fumarate and cyclopolymerisable monomers, aerylate and methacrylate esters, acrylic and methacrylic acid, styrene, acrylamide, methacrylamide, and methacrylonitrile, mixtures of these monomers, and mixtures of these monomers with other monomers.

Other examples of monomers of formula (IV) include: methyl methacrylate, ethyl methacrylate, propyl methacrylate (all isomers), butyl methacrylate (all isomers), 2-ethylhexyl methacrylate, isoborayl methacrylate, methacrylic acid, benzyl methacrylate, phenyl methacrylate, methacrylonitrile, alpha-methylstyrene, methyl aerylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, functional methacylates, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, vinyl benzoic acid (all isomers), diethylamino styrene (all isomers), alpha-methylvinyl benzoic acid (all isomers), diethylamino alpha-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilyl propyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyiacrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-phenylmaleimide, N-butylmaleimide, N-vinylpyrrolidone, N-vinylcarbazole, butadiene, ethylene and chloroprene. This list is not exhaustive.

Those skilled in the art will appreciate that ethylenically unsaturated monomer of general formula (III) may fall within the scope of ethyienically unsaturated monomer of general formula (IV). For the sake of clarity, where polymer used in accordance with the invention is described as being prepared from monomer of general formulae (III) and (IV) (i.e. where the so formed polymer is a copolymer), the scope of monomers to be selected from those of general formula (IV) are of course intended to exclude those that already fall within the scope of general formula (III).

In the context of general formulae (I) and (II), P_{A} and P_{B} each represent a polymer chain which may be the same or different, where each can independently comprise the first and/or second polymerised monomer residues, one or more other polymerised monomer residues, and combination thereof. For example, P_{A} and P_{B} can each represent a polymer chain which may be the same or different, where each can independently comprise polymerised residue of ethylenically unsaturated monomer selected from general formula (III), general formula (IV) and combinations thereof.

In one embodiment, the first polymerised residue of ethyienically unsaturated monomer has a structure represented in general formula (I) herein described where A is an ester, Sp is C₁₋₆alkyl and CAT is selected from guanidino and amidino.

In a further embodiment, polymer used in accordance with the invention further incorporates within its backbone structure a second polymerised residue of ethylenically unsaturated monomer, wherein the second polymerised residue has a structure represented in general formula (II) defined herein, where A is an ester, n is 0 and BIC is C₁₋₆alkyl.

In yet a further embodiment, the first polymerised residue of ethyienically unsaturated monomer has a structure represented in general formula (V):

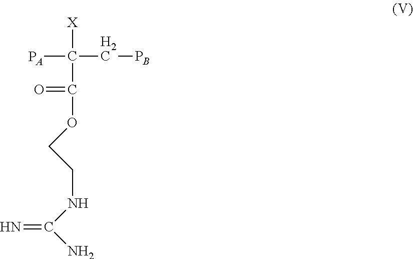

where P_{A}, P_{B} and X are as herein defined.

In another embodiment, the second polymerised residue of ethylenically unsaturated monomer has a structured represented in general formula (VI):

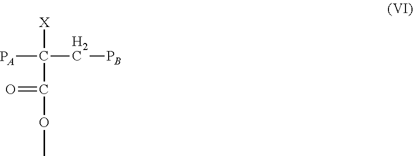

where P_{A}, P_{B} and X are as herein defined.

There is no particular limitation concerning the molecular weight of polymer used in accordance with the in vention. The number average molecular weight (Mn) of the polymer will generally range from about 2,000 to about 250,000, for example from about 3,000 to about 100,000. The Mn of the polymer is determined using gel permeation chromatography (GPC).

In one embodiment, polymer used according to the invention has adispersity (D) of less than 1.5, or less than 1.4, or less than 1.3, or less than 1.2.

As used herein, dispersity (Ð) of the polymer is determined according to equation (1):

Ð=M_{w}Mₙ (1)

where M_{w} is the mass average molecular weight, and Mₙ is as herein defined.

Polymer used in accordance with the invention may be prepared by a method comprising polymerising by free radical polymerisation ethyienically unsaturated monomer having a structure represented by general formula (III), optionally in combination with ethylenically unsaturated monomer of general formula (IV). Where ethylenically unsaturated monomer of general formula (IV) is used, it will of course have a structure not falling within the scope of monomers of general formula (III). According to the present invention the polymer must be prepared by polymerising at least monomer of general formula (III) where M is CAT. In other words, the so formed polymer must incorporate within its backbone structure the first polymerised residue of ethyienically unsaturated monomer.

In one embodiment, the first polymerised monomer residue is a 2-guanidinoethyl methacrylate residue.

In a further embodiment, the second polymerised monomer residue is a methyl methacrylate residue.

In yet a further embodiment, polymer used in accordance with the invention is a copolymer of 2-guanidinoethyl methacrylate and methyl methacrylate.

Polymer used in accordance with the invention may incorporate within its backbone structure from about 1 mol % to 100 mol % of the first polymerised residue (or general formula (I)), relative to the total amount of polymerised residue of ethyienically unsaturated monomer present. In one embodiment, the amount of the first polymerised residue (or general formula (I)) ranges from about 10 mol % to about 90 mol %, or from about 20 mol % to about 80 mol %, or from about 30 mol % to about 70 mol %, or from about 40 mol % to about 70 mol %, or from about 50 mol % to about 70 mol %, relative to the total amount of polymerised residue of ethylenieaily unsaturated monomer present.

When present, the second polymerised residue of ethylenieaily unsaturated monomer (or general formula (II)), will generally be incorporated, within the polymer backbone structure in an amount ranging from about 10 mol % to about 90 mol %, or from about 20 mol % to about 80 mol %, or from about 30 mol % to about 70 mol %, or from about 40 mol % to about 70mol %, or from about 50 mol % to about 70 mol %, relative to the total amount of polymerised residue of ethylenieaily unsaturated monomer present,

Similarly, when present polymerised residue derived from ethylenically unsaturated monomer of general formula (IV) will generally be incorporated within the polymer backbone structure in an amount ranging from about 5 mol % to about 60 mol %, or from about 5 mol % to about 50 mol %, or from about 5 mol % to about 40 mol %, or from about 5 mol % to about 30 mol. %, relative to the total amount of polymerised residue of ethylenieaily unsaturated monomer present.

Polymerisation of ethylenieaily unsaturated monomer by free radical polymerisation to prepare polymer used in accordance with the invention may require initiation from a source of free radicals. A source of initiating radicals can be provided by any suitable means of generating free radicals, such as the thermally induced hemolytic scission of suitable compound(s) (thermal initiators such as peroxides, peroxyesters, or azo compounds), the spontaneous generation from monomers (e.g. styrene), redox initiating systems, photochemical initiating systems or high energy radiation such as electron beam, X- or gamma-radiation.

Free radical polymerisation of monomer to form polymer used in accordance with the invention may proceed by conventional free radical polymerisation or by so-called living free radical polymerisation. Living polymerisation is generally considered in the art to be a form of chain polymerisation, in which irreversible chain termination is substantially absent. An important feature of living polymerisation is that polymer chains will continue to grow while monomer and the reaction conditions to support polymerisation are provided.

Where free radical polymerisation of the monomers occurs by a living polymerisation technique, it will generally be necessary to use a so-called living polymerisation agent. By “living polymerisation agent” is meant a compound that can participate in and control or mediate the living polymerisation of the ethylenically unsaturated monomers so as to form a living polymer chain (i.e. a polymer chain that has been formed according to a living polymerisation technique).

Examples of free radical living polymerisation techniques include iniferter polymerisation, stable free radical mediated polymerisation (SFRP), atom transfer radical polymerisation (ATRP), and reversible addition fragmentation chain transfer (RAFT) polymerisation.

In one embodiment, polymer used in accordance with the invention is prepared by iniferter polymerisation.

In a further embodiment, polymer used in accordance with the invention is prepared by SFRP.

In another embodiment, polymer used in accordance with the invention is prepared by ATRP.

In a further embodiment, polymer used in accordance with the invention is prepared by RAFT polymerisation.

Techniques, equipment and reagents for undertaking the free radical polymerisation of ethylenically unsaturated monomers are generally well known to those skilled in the an and can advantageously be applied in preparing polymer suitable for use in accordance with the present invention.

Polymer used in accordance with the invention can advantageously be prepared such that it is sufficiently soluble or readily dispersed in a carrier liquid so as to provide for a composition comprising an adequate amount of the polymer to adversely affect the biofilm. For example, the compositions in liquid form may comprise up to 1000 mg/L of polymer. In one embodiment, the compositions in liquid form comprise about 10 mg/L to about 500 mg/L of polymer, or from about. 50 mg/L to about 300 mg/L of polymer.

Polymer used in accordance with the invention has surprisingly and advantageously been found to function as a potent anti-microbial agent that adversely affects biofilms. The polymer may therefore also be described as being an anti-biofilm agent. From a practical point of view, the polymer, or composition comprising the polymer, used in accordance with the invention adversely affects the biofilm by inhibiting or preventing the biofilms growth, and/or by reducing the number of live microorganisms that form part of the biofilm. The biofilm may also be adversely affected by killing microorganisms that form part of the biofilm.

Accordingly, the invention provides a method of adversely affecting a biofilm, which includes inhibiting or preventing the growth of the biofilm, reducing the number of live microorganisms that form of the biofilm, and/or killing microorganisms that form part of the biofilm.

In one embodiment, at least. 10%, or at least 20%, or at least 30%, or at least 40%, or at least 50%, or at least 60%, or at least 70%, or at least 80% or at least 90% of microorganisms that form part of the biofilm are killed.

The method according to the invention is particularly suitable for adversely affecting a biofilm located on or in a subject. To that end, the invention also provides compositions suitable for administration to a subject, and also use of the polymer in the manufacture of a medicament for adversely affecting a biofilm.

According to the present invention, the biofilm is adversely affected by being exposed to the polymer or composition comprising the polymer described herein. Exposing the biofilm to the composition or polymer may be achieved by any suitable means known to those skilled in the art including coating, impregnating, spraying or otherwise contacting the biofilm, or a surface onto which the biofilm is adhered, with the polymer or polymer composition. Such exposure may occur as a result of administration of the composition or polymer to a subject comprising the biofilm.

There is no particular limitation on the nature or location of a biofilm that, can be adversely affected in accordance with the invention. For example, the biofilm may be located in or on a subject. The biofilm may also be located on a surface of an inanimate object such as a medical device.

In the context of administration to a subject, compositions in accordance with the invention may be applied to a bodily surface of the subject. That surface may be internal or external to the subject. For example, the biofilm may have been established on a human tissue surface (i.e. a biotic surface) such as a cornea or vitreous humor. Administration of the composition to the subject will of course be intended to bring about exposure of a biofilm in or on the subject to the polymer used in accordance with the invention. Administration may be achieved by any route considered suitable by those skilled in the art. Examples of routes of administration include those herein defined. Administration of the composition to a subject mav be performed once or more than once, including 2, 3, 4, 5 or more times, or as many times as required to achieve the desired outcome, and at any appropriate interval.

There is no particular limitation on the type of microorganism(s) that form the biofilm. For example, the biofilm may comprise, or consist essentially of, one or more microorganisms selected from Gram-positive bacteria such as Staphylococcus aureus, Staphylococcus epidermidis, Coagulase-negative Staphylococcus, Streptococcus sp., and mycobacterium tuberculosis, Gram-negative bacteria such as Klebsiella pneumoniae and Pseudomonas aeruginosa, and fungal pathogens such as Candida sp., and Candida albicans.

In one embodiment, the biofilm may comprise, or consist essentially of, one or more Gram-positive bacteria such as Staphylococcus aureus, Staphylococcus epidermidis, Coagulase-negative Staphylococcus, Streptococcus sp., and mycobacterium tuberculosis.

In another embodiment, the biofilm may comprise, or consist essentially of, one or both microorganisms selected from Candida albicans and Staphylococcus aureus.

The biofilm may be a monomicrobial or poly microbial biofilm. The method according to the invention has surprisingly been found to be particularly effective at adversely affecting polymicrobial biofilms.

In polymicrobial biofilms the mixed microbial species are present in a symbiotic relationship, sometimes benefiting each other. For example, beneficial interactions have been observed between S. aureus and C. albicans, such as higher microorganism load and increased antimicrobial resistance of the polymicrobial growth mode relative to single species biofilms. Also, when forming a polymicrobial biofilm C. albicans, S. aureus becomes more resistant to vancomycin and daptomycin than as a monoculture. Another bacterial species, Staphylococcus epidermidis, has been shown to protect C. albicans from the action of the antifungal drugs fluconazole and amphotericin B when growing together in polymicrobial biofilms. The enhanced drug resistance in polymicrobial biofilms has been attributed to components of the extracellular biofilm matrix secreted by fungi and bacteria.

In general, many current antimicrobial agents show poor efficacy against, biofilms, and in particular against polymicrobial biofilms. Polymer described and exemplified herein advantageously exhibits potent antimicrobial properties against both monomicrobial and against polymicrobial biofilms. The efficacy of these polymers against biofilms was only modestly decreased compared to planktonic growth, thereby suggesting that conventional biofilm-resistance mechanisms do not apply. Notably, the antimicrobial properties exhibited by the polymer can be derived using the polymer as the sole antimicrobial agent.

Without wishing to be limited by theory, it is believed the potent antimicrobial properties of the polymer is at least part derived from the polymer's molecular structure mimicking that of naturally occurring antimicrobial peptides (AMPs). AMPs have been identified as promising leads in the development of novel antibiotics due to their broad, spectrum antimicrobial activity, low toxicity towards human cells and a low susceptibility to current known mechanisms of resistance. However, their practical application is somewhat limited as the required proteins are typically pharmacokinetically unstable and large scale production is expensive. Polymer used in accordance with the invention advantageously presents a number of the desired properties of AMPs, but unlike AMPs they are stable, cheaper to produce and can be more readily chemically modified for tailored applications.

Polymer used in accordance with the invention has advantageously demonstrated low human cell toxicity.

While the methods and compositions in accordance with the invention are provided for adversely affecting a biofilm, their administration to a subject will typically be employed for the treatment, prevention and ongoing management of infectious diseases and of conditions, diseases and disorders associated with, characterised by, or caused by the presence of a biofilm in or on the subject. For example, microbial related infections associated with the presence of an established biofilm in or on a subject include infectious associated wife the use of medical devices including, but not limited to venous and urinary catheters, heart, valves and stents, prosthetic devices, tubings, implants (e.g. breast implants and intraocular lens), but also denture stomatitis, biofilm-related keratitis and infectious endophthalmitis.

Although polymer described and exemplified herein may be used as the sole antimicrobial agent for adversely affecting the biofilm, where desired the polymer may be used in combination with one or more other antimicrobial agents to adversely affect the biofilm.

In one embodiment, the present invention further comprises exposing the biofilm to a composition, comprising an effective amount of the polymer and one or more other antimicrobial agents. In that case, the exposure can be at the same time or at different times (i.e. the exposure can be simultaneous or sequential).

Where one or more other antimicrobial agents are employed, those agents can be co-formulated with the composition comprising the polymer or formulated in a separate composition. Where one or more other microbial agents are provided in different compositions, they can be administered to a subject or exposed to the biofilm in the same or different, routes or means as described herein.

Examples of suitable other antimicrobial agents include but are not limited to antifungal drugs (such as azoles, echinocandins, polyenes, flucytosine) and antibiotics such as penicillin (penicillin G), penicillinase-resistant β-lactam (oxacillin and methicillin), fluoroquinolones (ciprofloxacin), rifamycin (rifampicin), glycopeptide(vancomycin), and lipopeptide (daptomycin).

Formulation of compositions comprising the polymer used in accordance with the invention will of course depend on the application and/or administration technique employed. For example, a composition may be formulated in the form of a liquid, nasal spray, eyedrops, syrup, suspension, cream, powder, tablet, capsule, paste, lotion or gel.

Compositions comprising the polymer may further comprise one or more carriers, diluents or excipients.

In one embodiment, such carriers, diluents or excipients are pharmacologically acceptable. Examples of such suitable carriers, diluents or excipients include saline solution, deminerallzed or distilled water, mineral oil such as liquid paraffin, soft paraffin or squalane, vegetable based oil such as maze oil, olive oil, sesame oil, cotton seed oil, peanut oil safflower oil or coconut oil, silicone oils such as polysiloxanes, alcohols such as ethanol or isopropanol, polymers such as polyols such as polyethylene glycol, polypropylene glycol natural polymers such as alginate, starch and dextran glucan, gelatin or glycerine, fatty acid esters such as isopropyl palmitate, or ethyl oleate, agar, gum acacia, petroleum jelly, cellulose and alkyl cellulose derivatives, and solid substrates such as those made from metal, plastic, ceramic or combinations thereof.

The amount of polymer used in the methods or compositions in accordance with the invention will be an effective amount to achieve a desired result, which in the case of the present invention is to adversely affect a biofilm. The exact amount required to achieve the desired results will of course vary depending on factors such as a subject, or a situation under consideration, the composition of the biofilm, the volume or size of the biofilm to be exposed to the composition, the environment in which the biofilm is located and the means by which exposing the biofilm to the composition is conducted.

Where a composition comprising the polymer is provided in solid form for example in the form of a tablet or capsule, the polymer may be provided at a concentration ranging from about 0.01% (w/w) to about 60% (w/w), or from about 0.01% (w/w) to about 40% (w/w), or from about 0.5% (w/w) to about 20% (w/w), or from about 1% (w/w) to about 15% (w/w), or from about 1% (w/w) to about 10% (w/w).

Where the composition comprising the polymer is provided in a liquid form, the polymer may be provided in an amount ranging up to 1000 mg/mL. For example from about 10 mg/mL to about 1000 mg/mL, or from about 10 mg/mL to about 500 mg/mL, or from about 10 mg/mL to about 300 mg/mL, or from about. 50 mg/mL to about 300 mg/mL.

The most suitable concentration to employ may be readily determined by those skilled in the art using routine experimentation.

Where compositions used in accordance with the invention are formulated for administration to a subject, such as to treat an infections disease or condition associated with a biofilm, the composition will typically be administered to the subject in a therapeutically affective amount. Suitable dosage amounts and dosing regimes to achieve this can be determined by those skilled in the art and may depend on the particular condition being treated or diagnosed, the severity of the condition as well as the general age, health and weight of the subject Particular dosages can be empirically determined or extrapolated from, for example, studies in animals, or previous studies in humans. A suitable dosage of the polymer per se may lie within the range of about 0.1 ng per kg of body weight to 1 g per kg of body weight per dosage. The dosage may be in the range of 1 μg to 1 g per kg of body weight per dosage, such as is in the range of 1 mg to 1 g per kg of body weight per dosage. In one embodiment , the dosage may be in the range of 1 mg to 500 mg per kg of body weight, per dosage. In another embodiment, the dosage may be in the range of 1 mg to 250 mg per kg of body weight per dosage. In yet another embodiment the dosage may be in the range of 1 mg to 100 mg per kg of body weight per dosage, such as up to 50 mg per body weight per dosage.

Compositions in accordance with the invention may be administered in a single dose or a series of doses.

Compositions comprising polymer in accordance with the invention have been shown to function as effective antimicrobial lock solutions. Salvage of infected catheters and other implanted devices is important in clinical situations where removal of the device is not the preferred option. This can be achieved by so-called “antimicrobial lock therapy” (ALT), which entails treatment of the device with an antimicrobial agent to kill microorganisms that form the biofilm adhered to the device.

Ethanol ALT has been recommended as a first line option in device related bloodstream infections. However this is only recommended when the infection is caused by microorganisms with relatively low virulence such as coagulase-negative staphylococci (CoNS), or other microorganisms when device retention is warranted. The Infectious Diseases Society of America clinical guidelines recommend removal of the infected devices such as central venous catheters (CVC) in the case of S. aureus or C. albicans biofilm-related infections. Often the need for device salvage outweighs the risk, as device reinsertion might be difficult in patients with limited venous access and there is a high risk of morbidity and mortality associated with device replacement. Therefore an antimicrobial lock solution targeting biofilms is a valuable option to rescue the catheter. High concentrations of antifungal agents such as amphotericin B or caspofungin have been used in ALT and demonstrated activities against single Candida biofilms, but these would not be efficient with polymicrobial biofilms that also involve bacteria. Ethanol has been studied in vitro as a lock solution for catheter related bloodstream infections (CRBSI) caused by C. albicans or S. aureus individually. An in vitro study has shown that ethanol is effective as a lock solution against C. albicans-S. aureus polymicrobial biofilms. Although ethanol has a number of advantages, there are now concerns about its safety. Side-effects have been reported in regard to patient health, including systemic toxicity, headaches, lightheadedness, dizziness, arrhythmia, fatigue, nausea, and local venous irritation. Furthermore, the use of ethanol can lead to damage to the device, intraluminal obstruction and in some case thrombosis.

Compositions comprising polymer according to the invention may advantageously overcome one or more problems associated with using conventional lock solution.

Accordingly, the present invention further provides a method of performing antimicrobial lock therapy on a medical device having a biofilm adhered thereto, the method comprising exposing the biofilm to a composition comprising an effective amount of polymer that incorporates within its backbone structure a first polymerised residue of ethylenieaily unsaturated monomer, said first polymerised monomer residue comprising a covalently bound moiety that (i) presents pendant from the backbone structure, and (ii) comprises a cationic functional group or precursor functional group thereof.

The present invention further provides an antimicrobial lock solution for use in antimicrobial lock therapy, the composition comprising a pharmacologically acceptable carrier and polymer that incorporates within its backbone structure a first polymerised residue of ethylenieaily unsaturated monomer, said first polymerised monomer residue comprising a covalently bound moiety that (i) presents pendant from the backbone structure, and (ii) comprises a cationic functional group or precursor functional group thereof.

The aniimicrohiai lock solution in accordance with the invention will typically be used for performing antimicrobial lock therapy on a medical device ha ving a biofilm adhered thereto. Exposure of the biofilm to the antimicrobial lock solution advantageously adversely affects the biofilm as described herein.]

As used herein, the term “alkyI”, used either alone or in compound wonls denotes straight chain, branched or cyclic alkyl, preferably C₁₋₂₀alkyl, e.g. C₁₋₁₀ or C₁₋₆. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-methylpropyl, 1,1,2-trimethylpropyl, heptyl, 5-methyhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,13-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnontyl, 1-, 2-, 3-, 4-, 5- or 6-ethyloctyl, 1-, 2-. 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1,2-pentylheptyl and the like. Examples of cyclic alkyl include mono- or polycyclic alkyl groups such as cyclopropyl, cyclobutyl, cyclopentyl, cyclobesyl, cyclohcptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defiled.

The term “alkenyl” as used herein denotes groups formed from straight chain, branched or cyclic hydrocarbon residues containing al least one carbon to carbon, double bond including ethylenically mono-, di or polyunsaturated alkyl or cycloalkyl groups as previously defined, preferably C₂₋₂₀ alkenyl (e.g. C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cycloctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cyclobeptatrienyl and 1,3,5,7-cyclooctatetraenyl. An alkenyl group may be optionally substituted by one or more optional substituents as herein defined.

As used heroin the term “alkynyl” denotes groups formed from straight chain, branched or cyclic hydrocarbon residues containing at least one carbon-carbon triple bond including elhylenically mono-, di- or polyunsaturated alkyl or cycloalkyl groups as previously defined.

Unless the number of carbon atoms is specified the term preferably refers to C₂₋₂₀ alkynyl (e.g. C₂₋₁₀ or C₂₋₆). Examples include ethynyl, 1-propynyl, 2-propynyl, and butynyl isomers, and pentynyl isomers. An alkynyl group may be optionally substituted by one or more optional substituents as herein defined.

The term “halogen” (“halo”) denotes fluorine, chlorine, bromine or iodine (fluoro, chloro, bromo or iodo).

The term “aryl” (or “carboaryl”) denotes any of single, polynuclear, conjugated and fused residues of aromatic hydrocarbon ring systems (e.g. C₆₋₂₄ or C₆₋₁₈). Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may or may not be optionally substituted by one or more optional substituents as herein defined. The term “arylene” is intended to denote the divalent form of aryl.

The term “carbocyclyl” includes any of non-aromatic monocyclic, polycyelie, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cycloclenyl, cyclopentadienyl, cyclohexadienyl, cyclooctatetraenyl, indanyl, decalinyl and indenyl. A carbocyclyl group may be optionally substituted by one or more optional substituents as herein defined. The term “carbocyclylene” is intended to denote the divalent form of carbocyclyl.

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

The term “heterocyclyl” when used alone or in compound words includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. Particularly preferred heterecyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl mdoimyl, imidazolidinyl, imidazolinyl, pyraxolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxaxolidinyl, dihydropyranyl, oxazlnyl, thiazinyl, thiomorpholinyl, oxathianyl, dithianyl, trioxanyl, thiadiazmyl, dithiazinyl, trithianyl, azepinyl, oxepinyl, thiepinyl, iudenyl, indanyl, 3H-indolyl, isoindolinyl, 4H-quinolazinyl, ehromenyl, chromanyl, isochromanyl, pyranyl and dihydropyranyl. A heterocyclyl group may be optionally substituted by one or more optional substituents as herein defined. The term “heterocyclylene” is intended to denote the divalent form of heterocyclyl.

The term “heteroaryl” includes any of monocyclic, polycyclic, fused or conjugated hydrocarbon residues, wherein one or more carbon atoms are replaced, by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benxothienyl, isobeazothienyl, benzofuranyl, isobenzofuranyl, indolyl. isoindolyL pyrazolyl, pyrazinyl, pyrimidinyl, pyridazinyl, indolizinyl, quinolyl, isoquinolyl, phthalazinyl, 1,5-naphthyridinyl, quinoxalinyl, quinazolinyl, quinolinyl, oxazolyl, thiazolyl, isothiazolyl, isoxazolyl, triazolyl, oxadialzoyl, oxatriaxolyl, triazinyl and furazanyl. A heteroaryl group may be optionally substituted by one or more optional substiiuents as herein defined. The term “heteroarylene” is intended to denote the divalent form of heteroaryl.

The term “acyl” either alone or in compound words denotes a group containing the moiety C↑O (and not being a carboxylic acid, ester or amide) Preferred acyl includes C(O)—R^{e}, wherein R^{e} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, oetadecanoyl, nonadecanoyl and icosanoyl; cycloalkykarbonyl such as cyclopropylcarbonyl cyclobutylcarbonyl, cyclopentylcarbonyl and cyclohexycarbonyl; aroyl such as benzoyl, toluoyl and naphthoyl; aralkanoyl such as phenyl alkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. napthylpropenoyl, naphthylbutenoyl and naphthylpentenoyl; aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiaxolylacetyl, thiadiazolylacetyl and tetrazolylacetyl; heterocyclicalkenoyl such as hetcrocyclopropenoyl, heterocyclicbutenoyl, heterocyclicpentenoyl, and heterocychdiexenoyl; and heterocyclicglyoxyloyl such as thiazolyglyoxyloyl and thienylglyoxyloyl. The R^{e} residue may be optionally substituted as described herein.

The term “sulfoxide”, either alone or in a compound word, refers to a group —S(O)R^{f} wherein R^{f} is selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl.

The term “sulfonyl”, either alone or in a compound word, refers to a group S(O)₂—R^{f}, wherein R^{f} is selected from hydrogen, alkyl, alkenyl, alkynyl aryl, heteroaryl, heterocyclyl, carbocyclyl and aralkyl. Examples of preferred R^{f} include C₁₋₂₀alkyl, phenyl and benzyl.

The term “sulfonamide”, either alone or in a compound word, refers to a group S(O)NR^{f}R^{f} wherein each R^{f} is independently selected from hydrogen, alkyl, alkenyl, alkynyl, aryl, heteroaryl, heterocyclyl, carbocyclyl, and aralkyl. Examples of preferred R^{f} include C₁₋₂₀alkyl, phenyl and benzyl. In one embodiment at least one R^{f} is hydrogen. In another embodiment, both R^{f} are hydrogen.

The term, “amino” is used here in its broadest sense as understood in the art and includes groups of the formula NR^{a}R^{b} wherein R^{a} and R^{b} may be any independently selected from hydrogen, alkyl, alkenyl, alkynyl, aryl, carbocyclyl, heteroaryl heterocyclyl, arylalkyl, and acyl. R^{a} and R^{b}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋₂₀alkyl), NHaryl (e.g. NHphenyl), NHaralkyl (e.g. NHbenzyl), NHacyl (e.g. NHC(OC)C₁₋₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{a}R^{b}, wherein R^{a} and R^{b} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g, C(O)NHC(O)C₁₋₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term, “carboxy ester” is used here in its broadest sense as understood in the art and Includes groups having the formula CO₂R^{g}, wherein R^{g} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g., CO₂phenyl), CO₂aralkyl (e.g. CO₂benzyl).

As used herein, the term “aryloxy” refers to an “aryl” group attached through an oxygen bridge. Examples of aryloxy substituents include phenoxy, biphenyloxy, naphthyloxy and the like.

As used herein, the term “acyloxy” refers to an “acyl” group wherein the “acyl” group is in turn attached through an oxygen atom. Examples of “acyloxy” include hexylcarbonyloxy (heptanoyloxy), cyclopentylcarbonyloxy, benxoyloxy, 4-chlorobenxoyloxy, decylcarbonyloxy (undecanoyloxy), propylcarbonyloxy (butanoyloxy), octylcarbonyloxy (nonanoyloxy), biphenylcarbonloxy (eg 4-phenylbenzoyloxy), naphthylcarbonyloxy (eg 1-naphthoyloxy) and the like.

As used herein, the term “alkyloxycarbonyl” refers to a “alkyloxy” group attached through a carbonyl group. Examples of “alkyloxycarbonyl” groups include butylformate, sec-butylformate, hexylformate, octylformate, decylformate, cyclopentylformate and the like. As used herein, the term “arylalkyl” refers to groups formed from straight, or branched chain alkanes substituted with an aromatic ring. Examples of arylalkyl include phenylmethyl (benzyl), phenylethyl and phenylpropyl.

As used herein, the term “alkylaryl” refers to groups formed from aryl groups substituted with a straight chain or branched alkane. Examples of alkylaryl include methylphenyl and isopropylphenyl.

In this specification “optionally substituted” is taken to mean that a group may or may not be substituted, or fused (so as to form a condensed polycyclic group) with one, two, three or more of organic and inorganic groups, including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl nitrocarbocyclyl, nitroacyl, nitroalkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkythio, carbocyclylthio, heterocyclyl thio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioaikyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carhoxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidocyl, sulfonamidoaralkyl, nitroalkyl, nitroalkenyl, nitroalkynyl, nitrocarbocyclyl, nitroaryl, nitroheterocyclyl, nitroheteroaryl, nuroacyl, nitroaralkyl, cyano, sulfate, phosphate, triarylmethyl, triarylamino, oxadiazole, and carbazole groups. Optional substitution may also be taken to refer to where a —CH₂— group in a chain or ring is replaced by a group selected from —O—, —S—, —NR^{a}—, —C(O)— (i.e. carbonyl), —C(O)O— (i.e. ester), and —C(O)NR^{a}— (i.e. amide), where R^{a} is as defined herein.

Preferred optional substituents include alkyl, (e.g. C₁₋₆alkyl, such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆alkyl such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro alkyl, and amino), benzyl (wherein benzyl itself may be further substituted e.g., by C₁₋₆alkyl halo, hydroxy, hydroxyC₁₋₆alkyl, C₁₋₆alkoxy, haloC₁₋₆alkyl, cyano, nitro OC(O)C₁₋₆ alkyl, and amino), phenoxy (wherein phenyl itself may be further substituted e.g., by C₁₋₆alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkyl amino (e.g. C₁₋₆ alkyl, such as dimethylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by alkyl, halo, hydroxy, hydroxyC₁₋₆ alkyl, C₁₋₆ alkoxy, haloC₁₋₆ alkyl, cyano, nitro OC(O)CC₁₋₆ alkyl, and amino), nitro, formyl, —C(O)-alkyl (e.g. C₁₋₆ alkyl, such as acetyl), OC(O)-alkyl (e.g. C₁₋₆alkyl, such as acetyloxy), benzoyl (wherein the phenyl group itself may be further substituted e.g., by C₁₋₆alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C↑O, CO₂H, CO₂alkyl (e.g. C₁₋₆ alkyl such as methyl ester, ethyl ester, propyl ester, butyl ester), CO₂phenyl (wherein phenyl itself may be further substituted e.g., by C₁₋₆ alkyl, halo, hydroxy, hydroxyC₁₋₆ alkyl, C₁₋₆ alkoxy, halo C₁₋₆alkyl, cyano, nitro OC(O)C₁₋₆ alkylalkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl. (e.g. C₁₋₆ alkyl such as methyl ester, ethyl ester, propyl ester, butyl amide) CONHdialkyl (e.g. C₁₋₆ alkyl)aminoalkyl, (e.g., HN C₁₋₆alkyl-, C₁₋₆ alkylHN-C₁₋₆ alkyl- and (C₁₋₆ alkyl)₂N-C₁₋₆ alkyl-), thioalkyl (e.g., HS C₁₋₆ alkyl-), carboxyalkyl (e.g., HO₂CC₁₋₆ alkyl-), carboxyesteralkyl (e.g., alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆ alkyl(O)CC₁₋₆ alkyl-), nitroalkyl (e.g., O₂NC₁₋₆ alkyl-), sulfoxidealkyl (e.g., R(O)SC₁₋₆ alkyl, such as C₁₋₆alkyl(O)SCC₁₋₆alkyl-), sulfonylalkyl (e.g., R(O)₂SC₁₋₆ alkyl- such such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-), triaryhnethyl, triaryl amino, oxadiazole, and carbazole.

The invention will now be described with reference to the following non-limiting examples.

### EXAMPLES

Materials and Methods

Strains and Growth Condition

The wild-type C. albicans DAY 185 (ura3 Δ::λimm434/ura3- Δ::λimm434, ARG4:URA3:arg4::hisG/arg4::hisG his1::hisG::pHIS1/his1::hisG) and S. aureus ATCC 25923 were used as model microorganisms for C. albicans-S. aureus polymicrobial biofilms. Both strains are well-known monomicrobial biofilm-producers. C. albicans bgl2 homozygous mutant strain was obtained from the cell wall mutant library constructed by the laboratory of Dr. Aaron Mitchell. The BGL2 gene encodes a 1,3-beta-glucosyltransferase (Bgl2p). Lack of Bgl2p in C. albicans leads to deficiency in 1,3-beta-glucan levels in the biofilm matrix. Strains were stored at −80° C. in 15% (v/v) glycerol and streaked onto nutrient agar plates (NA, Oxoid, for S. aureus) or YPD plates (2% peptone, 1% yeast extract, 2% glucose, 80 mg/L uridine for C. albicans) as working stocks. The working stocks were stored at 4° C. (S. aureus) or room temperature (C. albicans) and replaced every two weeks.

Antimicrobial Agents

Four first-line antibiotics and three antifungals were used in these Examples: anti-staphylococcal agents included oxacillin (β-lactam), vancomycin (glycopeptide), ciprofloxacin (fluoroquinolone), rifampicin (rifamycin), and anti-candida agents included fluconazole (azote), amphotericin B (polyene), and caspofungin (echinocadin). All agents except caspofungin were purchased from Sigma-Aldrich, Sydney, Australia. Caspofungin was obtained from Merck & Co., Inc. Stock solutions of two guanylated polymethacrylates, designated as PG3 (50% MMA and 50% 2-GEMA content) and PG4 (32% MMA and 68% 2-GEMA), were prepared in distilled H₂O, as described in Polymer Chemistry 2014, 5, 5813-5822.

Biofilm Assays and Quantification

Bacterial, fungal and polymicrobial biofilm cultures were set up in 96-well microplates for quantification purposes and on silicon disks for microscopy. Briefly, overnight bacterial and fungal cultures were grown in nutrient broth (NB) and YPD broth respectively and diluted into growth medium, RPMI-1640, to a cell density of 1×10 ⁶ if CFU/mL. One hundred microlitres of the diluted bacterial, and/or fungal suspensions were pipetted into each well in a 96-well flat-bottom tissue culture treated polystyrene (TCPS) microplate and incubated for 24 h at 37° C. with gentle agitation (75 rpm). After incubation, the suspensions were aspirated and the microwells were rinsed twice with 110 μL of PBS per well to remove non-adherent cells. To quantitatively assess biofilm biomass, the microplate containing biofilms was heat-fixed in a 60° C. oven for 1 h and then stained with 1% (W/V) crystal violet (CV) for 10 min. The CV solution in the wells was then discarded and the microplates were washed four times to remove excessive stain. Two hundred microlitres of 95% ethanol plus 5% acetic acid were added into each well and the microplates were incubated at room temperature for 15 min. One hundred microlitres of the solutions from each well were transferred to a new microplate. The amount of biofilms formed was determined by reading the optical density with a Tecan Infinite M200 Plate Reader at 600 nm. Alternatively, after the microwells were rinsed with PBS to remove the non-adherent cells, 100 μL of PBS were added into each well and the biofilms were scrapped with sterile pipette tips. The microplate containing biofilms was then sonicated in a sooicaiioo hath (42 kHz) for 10 min. The bottom of the microwell was then scrapped again and the components in the microwell were transferred to an Eppendorf tube and vortexed for 30 s at maximum speed, four times. The suspensions were then serial diluted and plated on trypticase soy agar (TSA)+amphotericin B (2.5 mg/L) plates (to select for S. aureus) or YPD+ vancomycin (2 mg/L) plates (to select for C. albicans), to determine the cell numbers of each species in the biofilms.

Biofilm Microscopy

Bacterial, fungal and polymicrobial biofilm cultures were set up on silicon disks for microscopy purpose. Sterile silicon disks were pre-coated with adult bovine serum overnight prior to being transferred to a well in a 24 well raicropiate containing 1 mL of a S. aureus and/or C. albicans suspension in RPMI-1640 (˜10⁶ CFU/mL of each organisms), followed by overnight incubation at 37° C. The silicone disks were then rinsed three times with 0.9% saline to remove planktonic microorganisms. For scanning electron microscopy (SEM), biofilms were fixed with glutaraldehyde (2.5%, v/v) and 1% osmium tetraoxide at room temperature, and dehydrated with gradually increased concentrations of ethanol and hexamethyldisilazane (HMDS). Samples were coated with gold in a Balzers SCD005 sputter coater and viewed under a Hitachi S570 scanning electron microscope. For confocal laser scanning microscopy (CLSM), the biofilms were stained with BacLight Live/Dead Viability kit (L7007, Invitrogen) at 37° C. for 30 min in the dark after antimicrobial exposure. The structure of the biofilm was immediately examined after washing twice with 0.9% saline. To minimize artifacts associated with simultaneous dual wavelength excitation, all samples were sequentially scanned, frame-by-frame, first at 488 nm and then at 561 nm. A 63×oil objective was used in all imaging experiments.

Antimicrobial Susceptibility Tests for Planktonic Cells

Minimum inhibitory concentrations (MICs) for antibacterial and antifungal agents were determined using the broth microdilution method according to CLSI guidelines M07-A9 (for S. aureus) and M27-A3 (for C. albicans), but replacing Muller-Hinton broth with RPMI-1640 for antibacterial MIC testing. One hundred microlitres of two-fold serial dilutions of the drugs prepared in RPMI-1640 were added into the wells of 96-well microplates. Exponentially grown cultures were diluted in RPMI-1640 to a density of ˜1×10 ³ CFU/mL for C. albicans and ˜1×10⁶ CFU/mL for S. aureus and 100 μL were added to each well. Microplates were incubated for 18 h at 35° C. for S. aureus and 48 h for C. albicans. Bacterial or fungal growth was examined visually with the aid of a mirror reader. The antibacterial MIC was defined as the lowest concentration resulting in complete growth inhibition. The MIC of antifungals was read as the lowest concentration that prevents discernible growth for amphotericin 8 or polymers, or as the lowest concentration that inhibits at least 50% of fungal growth for fluconazole and caspofungin, corresponding to a score of zero or two in the CLSI M27-A3 protocol.

Antimicrobial Susceptibility Tests for Biofilm Cells

Single biofilms of S. aureus or C. albicans were set up in 96-well microplates as described above. After overnight incubation, the cell suspensions were aspirated and the wells were rinsed twice with 100 μL of PBS per well to remove non-adherent cells. Two hundred microlitres of antimicrobial agents were added into each well, and the treatments lasted for 18 h for S. aureus and 48 h for C. albicans. After the treatment, the suspensions were removed and the microwells were washed twice with PBS. Two hundred microlitres of 2,3-bis (2-methoxy-4-nitro-5-sulfophenyl)-5-[(phenylamino)carbonyl]-2H-tetrazolium hydroxide (XTT, 0.5 mg/L) solution were added into each well and the microplate were incubated at 37° C. in the dark for 2 h. One hundred microliters of XTT solution were transferred to a new microplate and read at OD₄₉₂. Alternatively, the efficacy of antimicrobial agents against biofilms was determined based on the reduction in viable counts after exposure to antimicrobials. The ratio of cell survival (cell density after antimicrobial treatment) relative to antimicrobial-free culture (cell density of the drug-free biofilms) (×100) was calculated.

Effects of Antimicrobial Combinations on Polymicrobial Planktonic Cultures And Biofilms

To determine the efficacies of combinations of conventional antimicrobials on polymicrobial biofilms, 100 μL volumes of RPMI-1640 alone or of RPMI-1640 containing one anti-staphylococcal agent and one anti-candida agent (twelve combinations in total) were added to established polymicrobial biofilms (Table 1). The antimicrobial concentrations were selected based on the highest serum achievable concentrations for intramuscular, intravenous or oral preparations (see Table 1). To avoid the paradoxical effect of caspofungin on C. albicans killing and inhibition, concentration of 4 mg/L was used for this drug to ensure its antifungal efficacy, replacing the highest serum achievable concentration of 12 mg/L. After incubating established polymicrobial biofilms with antimicrobial solutions or RPMT-1640 alone at 35° C. for 24 h, the solutions were aspirated from the wells and the biofilms were washed three times with PBS. Quantification of the biofilm survivor cells was carried out by the viable count method, as described previously. Differences between the cell counts from antimicrobial-treated and antimicrobial-free wells were used to calculate the efficacies of the antimicrobial combination against polymicrobial biofilms. In parallel, planktonic polymicrobial cultures of S. aureus (1×10⁶ CFU/mL) and C. albicans (1×10⁶ CFU/mL) were prepared and treated with the same set of antimicrobial combinations. Densities of 1×10⁶ CFU/mL were chosen for these microorganisms to provide a fair comparison between the planktonic cultures and biofilms, so that the same inocula of each species is challenged by the same amount of antimicrobial agents.

TABLE 1
Antimicrobial combinations and their
concentrations* used in this study

| | | Antifungals (mg/L) | | |
|---|---|---|---|---|
| Antibiotics | | | Amphotericin | |
| (mg/L) | + | Fluconazole or | B or | Caspofungin |
| Oxacillin | 16 | 8 | 2 | 4 |
| Vancomycin | 40 | 8 | 2 | 4 |
| Ciprofloxacin | 4.5 | 8 | 2 | 4 |
| Rifampicin | 10 | 8 | 2 | 4 |

*The highest serum achievable concentrations, for antibiotics were as referred in the Manual of Clinical Microbiology 10^{th} edition, for fluconazole in Brammer et al., 1990,⁵⁹ for amphotericin B in Clinical Infections Diseases: A Practical Approach, page 389. Concentration of 4 mg/L was used for caspofungin, replacing the highest serum achievable concentration of 12 mg/L to avoid its paradoxical effect.

Biofiim Matrix Isolation

Extracellular matrix was isolated from Candida or polymicrobial biofilms following the method reported in PLoS pathogens 2012, 8:e1002848. Biofilms were established bv growing cultures in 6-well TCPS microplates for 48 h at 37° C. Supernatants were carefully removed from each well and the biofilms were washed once with sterile distilled water. One millilitre of distilled water was added into each well, and the biofilms were removed using a cell scraper. This step was repeated three times. The suspensions containing dislodged biofilms were then transferred into a 50 ml Falcon tube, sonicated with a Branson Sonicator for 10 min (duty cycle constant, output 20%). The suspensions were vortexed for 2 min at full speed (4×30 s), prior to centrifugation at 6000 rpm for 15 min for 5 times. The superaatams containing the extracellular matrix were removed and stored at −20° C. Re-growth on TSA plates was examined to ensure the biofilm materials were free of viable cells.

To determine whether the C. albicans and polymicrobial biofilm matrix sequester the guanylated polymethacrylate polymers, the isolated matrix material was incubated with polymer and growth medium for 2 h in 96-well plates, prior to the addition C. albicans/S. aureus inocula for planktonic cell antimicrobial susceptibility testing. Results were compared to that of a standard susceptibility testing performed at the same time. Fluconazole was used as a positive control that demonstrates attenuated antimicrobial activities by C. albicans biofilm matrix, C. albicans biofilm matrix material, was also used as a supplement to RPMI-1640 to assess the impact, of C. albicans biofilm matrix on S. aureus biofilm growth.

In parallel, pre-formed overnight C. albicans biofilms by wild type DAY!85 and bgl2ΔΔ mutant strains were exposed to PG3 or PG4 at a sub-biofilm MIC (32 mg/L) or polymer-free growth medium for 24 h. C. albicans DAY 185 and bgl2ΔΔ mutant strains differ in the biofilm structure, with bgl2ΔΔ mutant strain forming a biofilm with less β-1,3 glucan in the extracellular matrix. Viable counts were performed. The efficacy of the polymers against biofilms was calculated as CFUs of biofilm treated with polymer divided by CFUs of biofilm exposed to polymer-free growth medium. The difference in the efficacy of polymers against wild type and mutant biofilms reflects the possible sequestering of the polymers by the biofilm matrix materials.

Gene Expression Analysts

C. albicans biofilms were grown in 6-well microplates in RPMI-1640 for 24 h and biofilm cells were collected. RNA was extracted using the hot-phenol method and contaminating genomic DNA was removed by treatment of total RNA with DNase I (Ambion). Reverse transcription was performed using Superscript III (Invitrogen), using 800 ng of Candida total RNA with 200 ng of mammalian total RNA as spike in control. Quantitative PGR was performed using the Fast-Start universal Sybr Green Master (Roche) on Roche Light Cycler LC 480 real-time PCR system, and analysed by absolute quantification. The expression levels of the transcripts were normalized first with mammalian GAPDH spike In control, followed by C. albicans SCR1 gene control. Four to eight independent biological replicates were used, with two technical replicates each. Sequences for all qPCR primers used in this study are listed In Table S1.

TABLE S1
Primers used for quantitative PCR

| Primers | Sequence (5′-3′) |
|---|---|
| mGAPDH | F: CTTTGTCAAGCTCATTTCCTG |
| | R: GCCATGAGGTCCACCACC |
| SCR1 | F: TTTAGCATAACCACTGGAGGGAAGC |
| | R: GAGTTGCAACACTAGATACCGCACT |
| BGL2/orf19.4565 | F: GCTGCTGAAGCTGAAGGATT |
| | R: GTGGAGACGGAAATCTTTGG |
| ALS1/orf19.5741 | F: ACCAATCCAGTTCCAACTGTGGCA |
| | R: TGGATGCTGATTCATGAGAACCGCT |
| ALS3/orf19.1816 | F: ACTTCCACAGCTGCTTCCACTTCT |
| | R: TCCACGGAACCGGTTGTTGCT |
| HWP1/orf19.1321 | F: AATCCTCCTCAACCTGATCAGCCTG |
| | R: AGCTGGAGTTGTTGGCTTTTCTGGA |
| EAP1/orf19.1401 | F: TGCCCCAGGTACTGAAACCACTC |
| | R: AGTGCCTGGGATAACGGGTTGAG |
| BCR1/orf19.723 | F: TACCTCCATTGCCACCAATACCTGC |
| | R: AGTGGTGGCTGTCCATGTTGTTGT |

Testing of the Efficacy of Polymers as Catheter Lock Solutions

A re-growth assay following catheter antimicrobial lock was performed to examine the efficacy of polymers used in accordance with the invention and ethanol as catheter lock solutions (CLS) against polymicrobial biofilms. Preformed 24-hour biofilms in 96 well microplates were exposed to antimicrobial agents for 1.8 h, including ethanol at concentrations ranging from 10% to 80%, and polymers of this invention, designated as PG3 and PG4 at 64-1024 mg/L. Biofilms were washed three times with saline to remove the residual antimicrobial agents. Two hundred microliter volumes of TSB were then added to each well and microplates were incubated at 35° C. for a further 48 h. A microplate shaker (speed 2) (Titreteck, Flow laboratories, Germany) was used to facilitate the multiplication and release of any living cells remaining in the biofilms. After 48 h, 150 μL of the contents in each well in the microplate was transferred to a U-bottom microplate and examined visually for turbidity. The lowest concentration of antimicrobial agents corresponding to clear wells was defined as the minimum biofilm eradication concentration (MBEC) for successful use as CLS.

Statistical Analysis

To analyze differences in cell numbers of biofilms formed by C. albicans and/or S. aureus, the data were transformed into a log 10 format and one-way ANOVA tests were performed with Minitab 16 for Windows using a significance level of 0.05 (p value).

Results

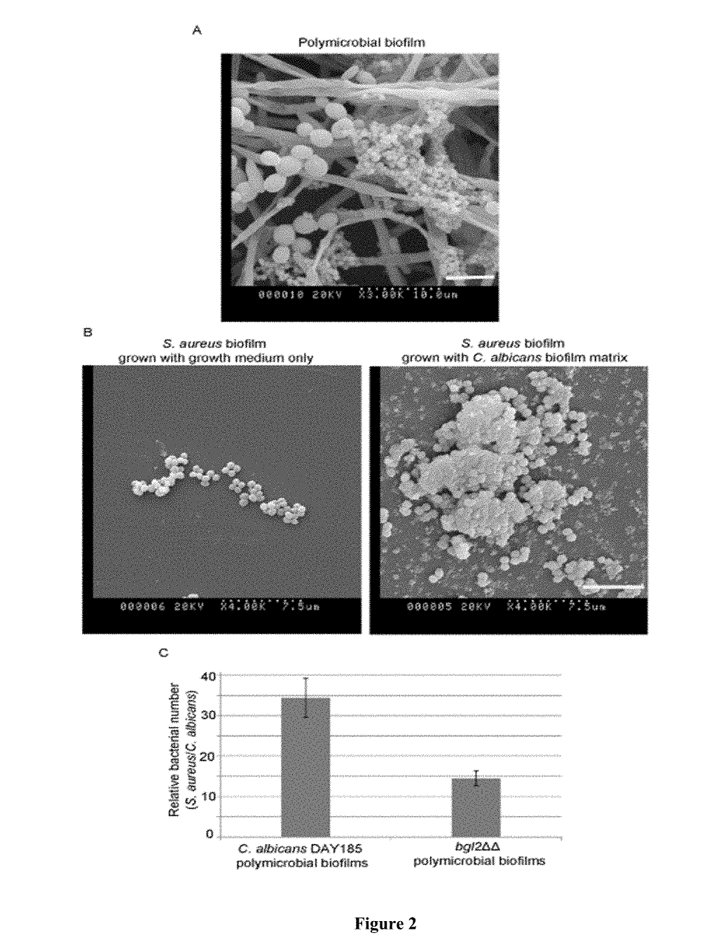

Role of a albicans biofilm matrix in the development of polymicrobial biofilms Association of S. aureus with C. albicans in a polymicrobial biofilm has been previously reported. Growth conditions and strain backgrounds are known to majorily affect biofilm. experiments, including polymicrobial C. albicans-S. aureus biofilm formation. The polymicrobial biofilms formed under the experimental conditions outlined here were therefore assessed, to ensure robust association of C. albicans and S. aureus. High-resolution SEM imaging of biofilms formed on biomedical silicone coated with adult bovine serum showed that S. aureus alone was able to form only a scarce biofilm under these conditions, but when incubated simultaneously with C. albicans the two species grew into a dense polymicrobial biofilm (FIG. 2A and B and FIG. S1A). Semi-quantitative CV-based biofilm measurements showed that the biomass of polymicrobial biofilms was higher than the sum of single biofilms (FIG. S1B). Cell number of S. aureus increased approximately twice when grown in polymicrobial biofilms relative to that of a single biofilm, while the number of C. albicans cells remained the same (Table S1). These polymicrobial biofilm characteristics am consistent with previous data, indicate robust polymicrobial biofilm growth under the experimental conditions outlined here. It was noticed that the presence of S. aureus rendered the polymicrobial biofilms more resistant to shear force (washing) and allowed the biofilm to attach more firmly to the microtitre plates or silicone disks, compared with single C. albicans biofilms. This indicates that not only does C. albicans help S. aureus association into the biofilm, but also the involvement of S. aureus provides the polymicrobial biofilm with a structural advantage, such as enhanced resistance to physical force.

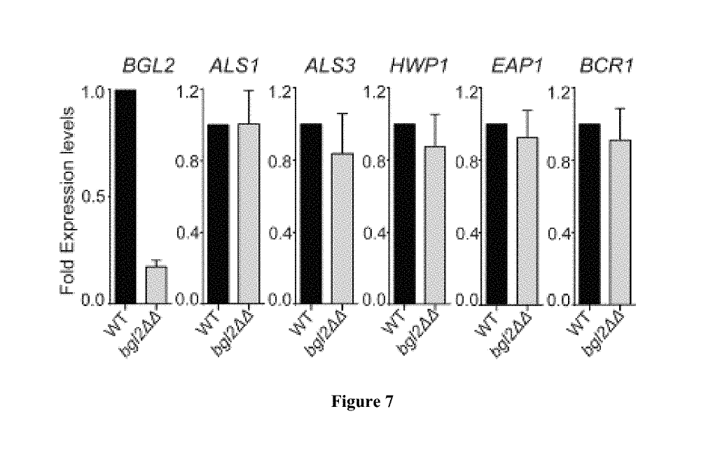

The interaction between S. aureus and C. albicans in a polymicrobial biofilm depends on the ability of C. albicans to form hyphae, and the hyphal surface protein Als3 is required for this interaction. In the polymicrobial biofilm, the presence of S. aureus was not limited to C. albicans hyphal cells some clumps of S. aureus were evident in the inter-cellular space of the biofilm scaffold (FIG. 2A). It is proposed that the C. albicans biofilm matrix could serve as another adhesion factor for S. aureus to integrate into the polymicrobial biofilm. Consistent with this proposition, single S. aureus biofilms grown in the presence of isolated matrix material from the C. albicans biofilm reached a significantly higher CFU count (1,5 fold) compared to that in the absence of C albicans biofilm matrix (Table 2). Scanning electron microscopy supported this conclusion, showing that in the presence of C. albicans biofilm matrix S. aureus could form significantly more pronounced biofilms than in the absence of matrix (FIG. 2B). It appeared that the matrix could attach to the serum coated silicone disc surface, and serve as a “glue” for the bacterium. Next, a C. albicans biofilm matrix mutant was used to test for the involvement of fungal biofilm matrix in S. aureus integration into the biofilm. C. albicans cell wall remodelling mutants that, display reduced biofilm matrix levels of β-1,3 glucan has been reported. One such mutant is inactivated in BGL2, the gene encoding a glocosyl-transferase involved in β-1,3 glucan remodelling (bgl2ΔΔ). Importantly, the bgl2ΔΔ mutant, is reported to form, a hyphae-rich biofilm, and the biofilm expression levels of ALS3, other hyphal specific cell wall, proteins and the biofilm regulator BCR1 did not change between wild type and bgl2ΔΔ biofilms (FIG. 7). To directly compare the ability of wild type and mutant C. albicans biofilms to incorporate S. aureus cells, the values were expressed as S. aureus CPUs per C. albicans cell. As shown in FIG. 2C, the polymicrobial biofilm scaffold formed by C. albicans bgl2ΔΔ strain attracted less S. aureus cells compared to wild type C. albicans DAY185 (FIG. 2C). This result supports the idea that extracellular biofilm matrix secreted by the fungus contributes to the association of S. aureus in the mixed biofilm.

TABLE 2
Cell enumeration of S. aureus biofilms grown
with/without C. albicans biofilm matrix

| −C. albicans biofilm matrix | +C. albicans biofilm matrix |
|---|---|
| (26.67 ± 8.5) × 10³ CFU | (40.67 ± 5.1) × 10³ CFU |

P = 0.003

Polymers in Accordance with the Invention as Effective Anti-Biofilm Agents that Outperform Current Antimicrobial Drugs and Drug Combinations

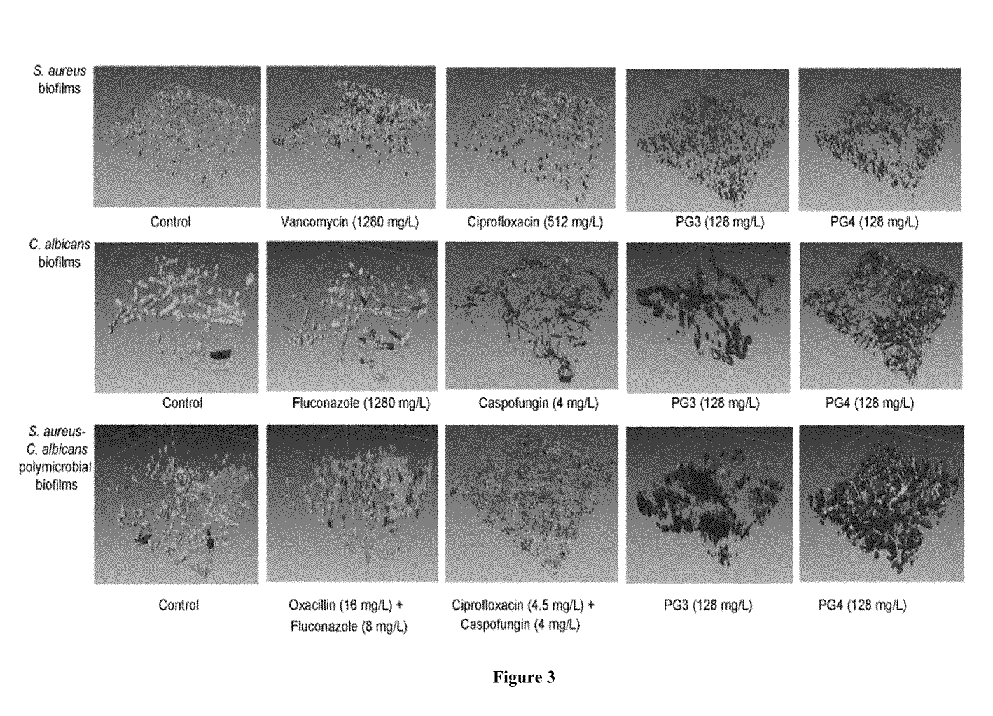

Consistent with numerous published studies, both S. aureus and C. albicans became highly resistant to most first-line antimicrobial agents when grown as monomicrobial biofilms, with only amphotericin B and caspofungin retaining their activities against C. albicans biofilms (Table 3). The two guanylated polymers used in the Examples, PG3 and PG4, demonstrated potent activities against S. aureus and C. albicans biofilms (Table 3). Biofilm MIC₈₀ was 128 mg/L against S. aureus and 64 mg/L against C. albicans, 16 and 4 times higher than their planktonic MICs respectively (Table 3). The efficacies of the guanylated polymers against single biofilms were confirmed by CLSM and BacLight Live/Dead staining for viable cells, showing that these polymers outperformed the current antifungal and antibacterial drugs (FIG. 3).

TABLE 3
Antimicrobial susceptibility of S. aureus and C. albicans
grown in different modes as a single species culture

| | S. aureus | | | C. albicans | |
|---|---|---|---|---|---|
| | Planktonic | Biofilm | | Planktonic | Biofilm |
| | MIC | MIC80 | | MIC | MIC80 |
| | (mg/L) | (mg/L) | | (mg/L) | (mg/L) |
| Oxacillin | 0.06 | >1024 | Fluconazole | 0.5 | >1024 |
| Vancomycin | 1 | >1024 | Amphotericin B | 1 | 1 |
| Ciprofloxacin | 0.25 | >1024 | Caspofungin | 1 | 0.12 |
| Rifaffipicin | 0.015 | >1024 | | | |
| PG3 | 8 | 128 | PG3 | 16 | 64 |
| PG4 | 8 | 128 | PG4 | 16 | 64 |

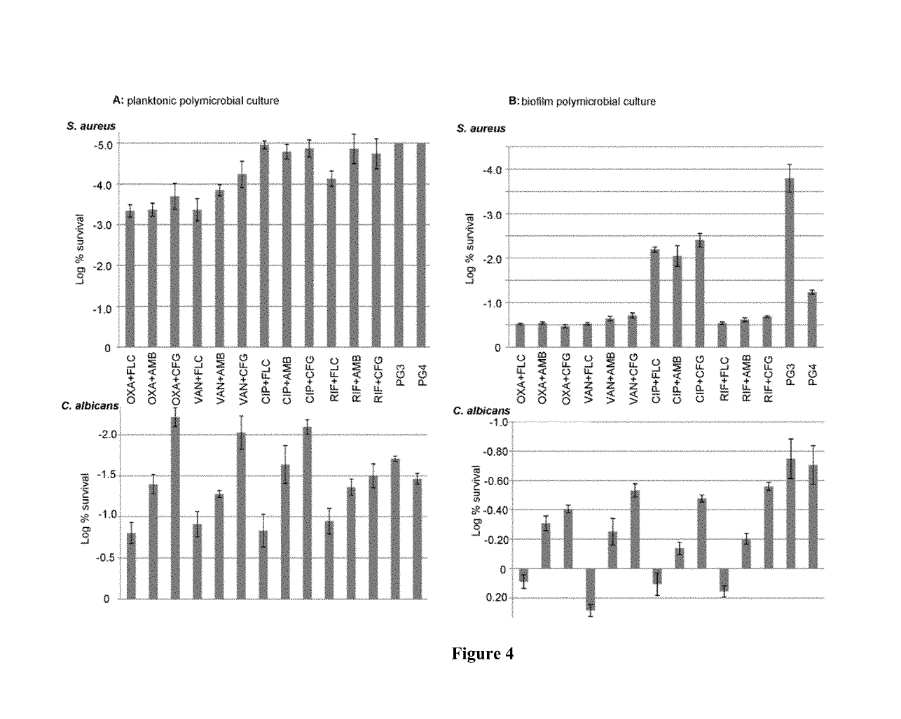

To further evaluate the potential of the guanylated polymers as anti-biofilm agents, their efficacy against polymicrobial biofilms was assessed, and their activity compared to combinations of conventional antibacterial and antifungal drugs. Firstly, we tested the susceptibility of C. albicans and S. aureus polymicrobial cultures to 12 antibacterial/antifungal combinations grown in a planktonic mode or biofilms respectively. The antimicrobials were used at the highest serum-achievable concentrations (see Table 1). Planktonic C. albicans-S. aureus polymicrobial cultures were susceptible to all 12 antibiotic/antifungal combinations (FIG. 4A), killing at least 99.9% of S. aureus and 90% of C. albicans (FIG. 4A). However, when challenging C. albicans-S. aureus polymicrobial biofilms, none of the 12 combinations except ciprofloxacin/caxpofungin demonstrated dual antimicrobial efficacies (FIG. 4B). The combination of ciprofloxacin and caspofungin was the best performer, reducing the S. aureus cell count by 99.6% and C. albicans by 66.6% (FIG. 4B). At the concentration of 128 mg/L PG3 and PG4 demonstrated a much more potent antibiofilm efficacy relative to conventional antimicrobial combinations (FIGS. 3 and 4B), PG3 killed 99.98% of S. aureus and 90% of C. albicans cells residing in the biofilm; PG4 killed 94% of S. aureus and 87% of C. albicans cells. We previously demonstrated that at this concentration the polymers display low-toxicity to human cells in vitro.

Biofiim Matrix Materials Sequester Polymer in Accordance with the Invention, but with Minor Effects on Susceptibility

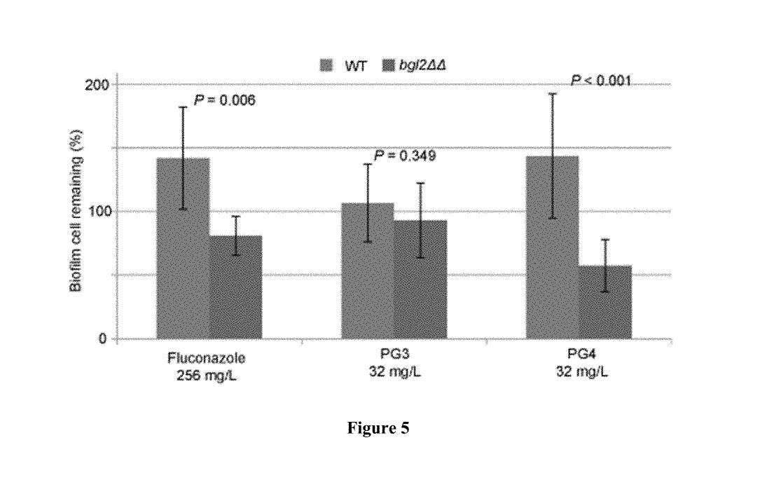

A major contributor to the increased antimicrobial resistance of biofilms (bacterial fungal and polymicrobial) is the extracellular matrix material. The biofilm MICs for the guanylated polymers used in accordance with the invention were only modestly higher than planktonic MICs, but we reproduelbiy observed this difference, suggesting that, the biofilm growth mode confers some resistance to these compounds. To test whether the extracellular biofilm matrix mediates this effect, we tested the antimicrobial susceptibility of the guanylated polymers against planktonic S. aureus and C. albicans cells grown in the presence of C. albicans biofilm matrix or polymicrobial biofilm matrix. Under this condition, fluconazole, a positive control agent, showed a 16-fold increase in its MIC against C. albicans. Minimum inhibitory concentrations of PG3 and PG4 against S. aureus and C. albicans also increased by 4 fold and 2 fold respectively (Table 4). These increases in MICs are minor, but they were highly 5 reproducible. Elevated MIC levels suggested that either the extracellular biofilm material suppresses the activities of these polymers, or the biofilm matrix promotes the adherence of planktonic cells to the substratum and formation, of “more resistant” monolayers. To further clarify this, we used the C. albicans bgl2ΔΔ mutant strain, which displays lower levels of β-1,3 glucan in the biofilm extracellular matrix and is more sensitive to fluconazole. To perform this assay, we used a sub-biofilm MIC for PG3 and PG4 (32 mg/L) to detect any difference between wild type biofilms and the bgl2ΔΔ mutant biofilms. As shown in FIG. 5, lowered β-1.3 glucan in the biofilm extracellular matrix led to significantly increased efficacy of PG4 against C. albicans biofilms, while there was no effect on PG3 (FIG. 5).

TABLE 4
Antimicrobial susceptibility of S. aureus and C. albicans in the presence or absence of biofilm matrix (MICs)

| | S. aureus (mg/L) | | | | C. albicans (mg/L) | | |
|---|---|---|---|---|---|---|---|
| | Medium | C. albicans | Polymicrobial | | Medium | C. albicans | Polymicrobial |
| | only | biofilm matrix | biofilm matrix | | only | biofilm matrix | biofilm matrix |
| Vancomycin | 2 | 1 | 2 | Fluconazole | 0.5 | 8 | 8 |
| PG3 | 8 | 32 | 32 | PG3 | 16 | 32 | 32 |
| PG4 | 8 | 32 | 32 | PG4 | 16 | 32 | 32 |

Polymers in Accordance with the Invention as Antimicrobial Lock Solutions

Salvage of infected catheters is important in clinical situations where catheter removal is not the preferred option. This can be achieved by the so-called “antimicrobial lock therapy” or ALT, which entails treatment of the catheter with antimicrobial agents to kill the infecting organisms. Ethanol has been reported as an effective lock solution against C. albicans-S. aureus polymicrobial biofilms, but its application in ALT has been hindered by side effects. We show here that one application of the invention is a catheter lock agent. Polymicrobial biofilms were formed in 96-well microplates, followed by application of ethanol or guanylated polymethacrylates to biofilms and overnight exposure. As shown in Table 5, PG3 at 128 mg/mL or PG4 at 256 mg/L were effective in salvaging biomaterials infected by polymicrobial biofilms, effectively eradicating C. albicans-S. aureus biofilms. This effect was comparable to exposure to 20% ethanol overnight (Table 5), PG3 and PG4 at minimum biofilm eradication concentration (MBEC) display very low human cell toxicity and might be used as an alternative to ethanol for catheter lock solutions.

TABLE 5
MBECs of polymers as catheter lock solution
against 24 h single/polymicrobial biofilms

| | Ethanol (V/V) | PG3 (mg/L) | PCM (mg/L) |
|---|---|---|---|
| S. aureus | 20% | 128 | 256 |
| C. albicans | 20% | 128 | 256 |
| Polymicrobial | 20% | 128 | 256 |

Polymers in Accordance with the Invention as Chronic Wound Dressings

The polymers can be formulated according to standard pharmaceutical protocols to provide stable/effective dosing of polymers to wound. Typically they could be disperse within an appropriate matrix to provide sustained release of the polymer to the wound over time. Typical matrix could include alginates, chitosan, starches, dextran, glucan, gelatin to name a few.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of adversely affecting a an established biofilm, the method comprising exposing the biofilm to a composition comprising an effective amount of copolymer that incorporates within its backbone structure a first polymerised residue of ethylenically unsaturated monomer, said first polymerised monomer residue comprising a covalently bound moiety that (i) presents pendant from the backbone structure, and (ii) comprises a cationic functional group or precursor functional group thereof.

2. The method according to claim 1, wherein the copolymer further incorporates within its backbone structure a second polymerised residue of ethylenically unsaturated monomer, said second polymerised monomer residue comprising a covalently bound hydrophobic moiety that presents pendant from the backbone structure.

3. The method claim 1, wherein the cationic functional group or precursor functional group thereof comprises a nitrogen or phosphorous atom.

4. The method of claim 1, wherein the cationic functional group or precursor functional group thereof is selected from the group consisting of primary amine, secondary amine, tertiary amine, quaternary amine, and quaternary phosphine.

5. The method of claim 1, wherein the cationic functional group or precursor functional group thereof is selected from guanidino and amidino.

6. The method according to claim 2, wherein the covalently bound hydrophobic moiety is selected from an alkyl or aryl moiety.

7. The method of claim 1, wherein the biofilm comprises one or more microorganisms selected from the group consisting of Staphylococcus aureus, Staphylococcus epidermidis, Coagulase-negative Staphylococcus, Streptococcus sp., mycobacterium tuberculosis, Klebsiella pneumoniae Pseudomonas aeruginosa, Candida sp., and Candida albicans.

8. The method of claim 1, wherein the biofilm is a polymicrobial biofilm.

9. The method of claim 1, wherein the polymer is a guanylated polymethacrylate.

10. The method of claim 1, wherein the copolymer is a random copolymer of 2-guanidinoethyl methacrylate or methyl methacrylate.

11. The method of claim 1, wherein the biofilm comprises one or both microorganisms selected from Candida albicans and Staphylococcus aureus.

12. The method of claim 1, wherein as a result of the biofilm being exposed to the composition microorganisms that form part of the biofilm are killed.

13. A method of adversely affecting a an established biofilm located on or in a subject, the method comprising exposing the biofilm to a composition comprising an effective amount of copolymer by administering the composition to the subject, wherein the polymer incorporates within its backbone structure a first polymerised residue of ethylenically unsaturated monomer, said first polymerised monomer residue comprising a covalently bound moiety that (i) presents pendant from the backbone structure, and (ii) comprises a cationic functional group or a precursor group thereof.

14-18. (canceled)


SEQUENCE LISTING (18 sequences)

SEQ ID NO: 1 - DNA, 21 bp, homo sapien
ctttgtcaagctcatttcctg

SEQ ID NO: 2 - DNA, 18 bp, homo sapien
gccatgaggtccaccacc

SEQ ID NO: 3 - DNA, 25 bp, C. albicans
tttagcataaccactggagggaagc

SEQ ID NO: 4 - DNA, 25 bp, C. albicans
gagttgcaacactagataccgcact

SEQ ID NO: 5 - DNA, 20 bp, C. albicans
gctgctgaagctgaaggatt

SEQ ID NO: 6 - DNA, 20 bp, C. albicans
gtggagacggaaatctttgg

SEQ ID NO: 7 - DNA, 20 bp, C. albicans
gctgctgaagctgaaggatt

SEQ ID NO: 8 - DNA, 20 bp, C. albicans
gtggagacggaaatctttgg

SEQ ID NO: 9 - DNA, 24 bp, C. albicans
accaatccagttccaactgtggca

SEQ ID NO: 10 - DNA, 25 bp, C. albicans
tggatgctgattcatgagaaccgct

SEQ ID NO: 11 - DNA, 24 bp, C. albicans
acttccacagctgcttccacttct

SEQ ID NO: 12 - DNA, 21 bp, C. albicans
tccacggaaccggttgttgct

SEQ ID NO: 13 - DNA, 25 bp, C. albicans
aatcctcctcaacctgatcagcctg

SEQ ID NO: 14 - DNA, 25 bp, C. albicans
agctggagttgttggcttttctgga

SEQ ID NO: 15 - DNA, 23 bp, C. albicans
tgccccaggtactgaaaccactc

SEQ ID NO: 16 - DNA, 23 bp, C. albicans
agtgcctgggataacgggttgag

SEQ ID NO: 17 - DNA, 25 bp, C. albicans
tacctccattgccaccaatacctgc

SEQ ID NO: 18 - DNA, 24 bp, C. albicans
agtggtggctgtccatgttgttgt